(19) United States
(12) Patent Application Publication
(10) Pub. No.: US 2025/0281675 A1
(43) Pub. Date: Sep. 11, 2025

(54) SOFT TISSUE COMPOSITE

(71) Applicant: LifeNet Health, Virginia Beach, VA (US)

(72) Inventors: Xiaofei Qin, Virginia Beach, VA (US); Austin Johnson, Virginia Beach, VA (US); Erin Cresswell, Virginia Beach, VA (US); Michael Valentine, Virginia Beach, VA (US); Bethany Young, Virginia Beach, VA (US); Tak Lung Chang, Virginia Beach, VA (US); Jingsong Chen, Virginia Beach, VA (US)

(21) Appl. No.: 18/859,463

(22) PCT Filed: Apr. 26, 2023
(86) PCT No.: PCT/US2023/020083

§ 371 Date: Oct. 23, 2024

Related U.S. Application Data
(60) Provisional application No. 63/334,971, filed on Apr. 26, 2022.
(60) Provisional application No. 63/430,883, filed on Dec. 7, 2022.
(60) Provisional application No. 63/457,270, filed on Apr. 5, 2023.

(51) Int. Cl. A61L 27/48; A61L 27/18; A61L 27/24; A61L 27/26; A61L 27/36; A61L 27/56

(52) U.S. Cl. CPC A61L 27/48; A61L 27/18; A61L 27/24; A61L 27/26; A61L 27/3604; A61L 27/3641; A61L 27/56; A61L 27/3691; A61L 2400/06; A61L 2430/34

(57) ABSTRACT

The present invention provides a soft tissue composite. The soft tissue composite comprises a first collagen fiber bundle of a first soft tissue isolated from a first donor and a polymeric material, and the first collagen fiber bundle interconnects with the polymeric material. The soft tissue may be a sandwich composite comprising a first layer comprising a first collagen fiber bundler, a second layer comprising a polymeric material, and a third layer comprising a third collagen fiber bundles, and the second layer is between the first and third layers, and the first collagen fiber bundler, the polymeric material and the third collagen fiber bundler interconnect with each other. Also provided is a method of repairing a defective tissue in a subject with the soft tissue composite and a method of preparing the soft tissue composite.

### CROSS-REFERENCE TO RELATED APPLICATION

This application claims priority to U.S. Provisional Application No. 63/334,971, filed Apr. 26, 2022, Provisional Application No. 63/430,883, filed Dec. 7, 2022, and Provisional Application No. 63/457,270, filed Apr. 5, 2023, and the contents of each of which are incorporated herein by reference in their entireties for all purposes.

### FIELD OF THE INVENTION

The invention relates generally to a soft tissue composite, and uses thereof in repairing a defective tissue which may be weaken due to injury or disease.

### BACKGROUND OF THE INVENTION

Various soft tissue products have been developed in a variety of forms. Intended uses of soft tissue products may dictate certain aspects of its form such as size, shape, or thickness. Biologically functional scaffolds having a porous structure have been developed for repair of a defective tissue. There remains a need for a soft tissue composite of a soft tissue scaffold and a synthetic material with desirable biological and physical properties that may be employed for Minimally Invasive Surgery (MIS) procedures and repair of a defective tissue in a subject.

### SUMMARY OF THE INVENTION

The present invention relates to a soft tissue composite and uses and preparation thereof.

The present invention provides a soft tissue composite. The soft tissue composite comprises a first collagen fiber bundle of a first soft tissue isolated from a first donor and a polymeric material. The first collagen fiber bundle interconnects with the polymeric material.

The polymeric material may be entangled with the first collagen fiber bundle. The polymeric material comprises pores, and the first collagen fiber bundle may pass through the pores.

The soft tissue composite may lack a chemical crosslinker. The soft tissue composite may lack a non-naturally occurring crosslinker.

The first collagen fiber bundle may not have a surface treatment.

The soft tissue composite may have a tear resistance of 5-200 N.

The soft tissue composite may maintain at least 80% of its mechanical strength for at least 30 days in vivo.

The first soft tissue may be selected from the group consisting of muscle, fat, blood vessel, nerve, tendon, ligament, lining of joints, skin, dermis, pericardium, fascia, cartilage, dura mata, endocardium, mucosal tissue, placental membrane, periosteum, bladder, small intestine, large intestine, urethra, and placenta.

The first collagen fiber bundle may have been frozen, freeze-dried, and/or plasticized.

The first collagen fiber bundle may be selected from the group consisting of a first split collagen fiber bundle, a first dispersed collagen fiber bundle and a combination thereof. The first dispersed collagen fiber bundle may be in the form of a first soft tissue scaffold or a first collagen yarn.

The polymeric material may be selected from the group consisting of a synthetic polymeric material, a natural polymeric material, and a combination thereof.

The polymeric material may be in the form of a polymer strand, a polymer ribbon or a polymer mesh.

The polymeric material may comprise one or more biodegradable polymers selected from the group consisting of polylactic acid, polyglycolic acid, poly-4-hydroxybutyrate, polydioxanone, trimethylene carbonate, polycaprolactone, silk, silk fibroin, and a combination thereof.

The first collagen fiber bundle may be a first split collagen fiber bundle. The polymeric material may be in the form of a polymer strand or a polymer ribbon, and the split first collagen fiber bundle and the polymer strand or polymer ribbon may form a composite strand or ribbon. The soft tissue composite may further comprise an additional collagen fiber bundle.

The first collagen fiber bundle may be a first dispersed collagen fiber bundle. The polymeric material may be in the form of a polymer strand or polymer ribbon, and the first dispersed collagen fiber bundle and the polymer strand or polymer ribbon may form a composite strand or ribbon. The first dispersed collagen fiber bundle may be in the form of a first collagen yarn. The polymeric material may be in the form of a polymer mesh, and the first dispersed collagen fiber bundle and the polymer mesh may form a composite scaffold. The soft tissue composite may further comprise an additional collagen fiber bundle.

The first collagen fiber bundle may be in the first isolated soft tissue. The polymeric material may be in the form of a polymer strand or polymer ribbon, and the first isolated soft tissue and the polymer strand or polymer ribbon may form a composite strand or ribbon or a composite scaffold. The soft tissue composite may further comprise an additional collagen fiber bundle.

The first collagen fiber bundle may be a first dispersed collagen fiber bundle in the form of a first soft tissue scaffold. The polymeric material may be in the form of a polymer strand or polymer ribbon, and the first soft tissue scaffold and the polymer strand or polymer ribbon may form a composite strand or ribbon or a composite scaffold. The soft tissue composite may further comprise an additional collagen fiber bundle.

The additional collagen fiber bundle may be an additional dispersed collagen fiber bundle in the form of an additional soft tissue scaffold, and the additional soft tissue scaffold and the composite strand or ribbon may form a composite scaffold.

The composite strand or ribbon may form a composite mesh, and the additional dispersed collagen fiber bundle and the composite mesh may form a composite scaffold.

The soft tissue composite may comprise a first layer, and the first layer may comprise the first collagen fiber bundle. The first collagen fiber bundle may be a first dispersed collagen fiber bundle in the form of a first soft tissue scaffold, and the polymeric material may be a synthetic material. The soft tissue scaffold may be pressed. The soft tissue composite may be dry and flexible. The synthetic material may pass through the first soft tissue scaffold. The synthetic material may be in the form of a polymer strand or ribbon. The first soft tissue scaffold and the synthetic material may form a braided or wrapping pattern.

The soft tissue composite may further comprise a second layer, and the second layer may comprise the polymeric material in the form of a polymer mesh. The first layer may cover the second layer. The second layer may further comprise a natural polymeric material.

The polymer mesh may have a thickness of 0.01-10 mm.

The polymer mesh may have a projection or height of 2-8 cm.

The polymer mesh may have a thickness no less than 0.01 mm and a width or diameter of 7-20 cm.

The polymer mesh may have a thickness no greater than 5 mm and a width or diameter of 0.1-100 cm.

The polymer mesh may have a thickness no greater than 5 mm and a length of 1-500 cm.

The polymer mesh may have an average pore size from 2 mm to 20 mm and an average of pore area from 4 mm² to 400 mm².

The polymer mesh may have a flexibility rigidity from 1 μNm to 200 μNm.

The polymer mesh may have a drape coefficient of 50-5%.

The polymer mesh may have a suture retention strength in the range of 5-500 N.

The polymer mesh may have a tear resistance of 5-200 N.

The polymer mesh may have an absorption time in vivo from 30 days to 18 months.

The polymer mesh may maintain at least 80% of its mechanical strength for at least 30 days in vivo.

The soft tissue composite may further comprise a third layer, the second layer may be between the first layer and the third layer, and the third layer may comprise a third collagen fiber bundle of a third soft tissue isolated from a third donor. The third collagen fiber bundle may be in the form of a third soft tissue scaffold. The polymer mesh may have pores, and the first soft tissue scaffold and the third soft tissue scaffold may interconnect with each other through the pores. The polymeric material may be entangled with the third collagen fiber bundle. The first soft tissue scaffold and the third soft tissue scaffold may be different. The first soft tissue scaffold and the third soft tissue scaffold may be identical. The third soft tissue may be selected from the group consisting of muscle, fat, blood vessel, nerve, tendon, ligament, lining of joints, skin, dermis, pericardium, fascia, cartilage, dura mata, endocardium, mucosal tissue placental membrane, periosteum, bladder, small intestine, large intestine, urethra, and placenta. The third collagen fiber bundle may have been frozen, freeze-dried, and/or plasticized. The second layer may further comprise a second collagen fiber bundle of a second soft tissue isolated from a second donor. The second collagen fiber bundle may be a second dispersed collagen fiber bundle. The second dispersed collagen fiber bundle may cover the synthetic material.

The soft tissue composite may be dry.

The soft tissue composite may be pressed.

The first layer and the second layer may be stitched together.

The first layer, the second layer and the third layer may be stitched together.

The polymeric material may comprise a first polymer, a second polymer, and a third polymer. The first polymer, the second polymer, and the third polymer may be identical. The first polymer, the second polymer, and the third polymer may be different. The second polymer may be connected to the first polymer.

The soft tissue composite may further comprise a surgical suture.

The soft tissue composite may be hydrated. The hydrated soft tissue composite may be pressed by force. The pressed and hydrated soft tissue composite may be capable of regaining the original shape and thickness after the pressing force is removed.

For each soft tissue composite of the present invention, a medical device comprising the soft tissue composite is provided. The medical device may further comprise a breast implant, breast reconstruction, pacemaker implant, microchip implant, metal bone implant, a drug delivery device implant, internal monitor implant, allograft tissue implantation, autograft tissue implantation, device implant, bolster flap transfer, vascular graft, stent, or a combination thereof.

A method of repairing a defective tissue in a subject in need thereof is provided. The repairment method may comprise administering the soft tissue composite or the medical device of the present invention to the defective tissue in the subject. The repairment method may further comprise suturing or stapling the soft tissue composite to the defective tissue. The soft tissue composite or the medical device may be administered to the subject via a delivery device. The delivery device may be a syringe or endoscopic clip. Where the soft tissue composite is dry, the repairment method may further comprise hydrating the dry soft tissue composite before the administering.

For each soft tissue composite of the present invention, a preparation method of preparing the soft tissue composite is provided. The preparation method comprises interconnecting the first collagen fiber bundle with the polymeric material. The interconnecting may lack chemical crosslinking.

Where the first collagen fiber bundle is a first split collagen fiber bundle, the preparation method may further comprise splitting the first soft tissue isolated from the first donor to make the first split collagen fiber bundle.

Where the first collagen fiber bundle is a first dispersed collagen fiber bundle, the preparation method may further comprise dispersing the first soft tissue to make the first dispersed collagen fiber bundle.

Where the first dispersed collagen fiber bundle is in the form of a first soft tissue scaffold, the preparation method may further comprise molding the first dispersed collagen fiber bundle into the first soft tissue scaffold.

Where the first dispersed collagen fiber bundle is in the form of a first collagen yarn, the preparation method may further comprise twisting, spinning, and/or pressing the first dispersed collagen fiber bundle into the first collagen yarn.

Where the polymeric material is in the form of a polymer strand or polymer ribbon, the preparation method may further comprise extruding and/or spinning the polymeric material into the polymer strand or polymer ribbon.

Where the polymeric material is in the form of a polymer mesh, the preparation method may further comprise knitting, weaving, or crocheting the polymer strand or ribbons into a polymer mesh.

Where the polymeric material is in the form of a polymer mesh, the preparation method may further comprise printing the polymeric material into a polymer mesh.

Where the first collagen fiber bundle is a first split collagen fiber bundle and the polymeric material is in the form of a polymer strand or polymer ribbon, the preparation method may further comprise braiding the first split collagen fiber bundle with the polymer strand or polymer ribbon to form a composite strand or ribbon.

Where the first collagen fiber bundle is a first dispersed collagen fiber bundle and the polymeric material is in the form of a polymer strand or polymer ribbon, the preparation method may further comprise coating and/or braiding the first dispersed collagen fiber bundle with the polymer strand or polymer ribbon to form a composite strand or ribbon. The first dispersed collagen fiber bundle may be in the form of a collagen yarn.

Where the first collagen fiber bundle is in the first soft tissue and the polymeric material is in the form of a polymer strand or polymer ribbon, the preparation method may further comprise stitching and/or sewing the first soft tissue with the polymer strand or polymer ribbon to form a composite strand or ribbon.

Where the first collagen fiber bundle is in the form of a first soft tissue scaffold and the polymeric material is in the form of a polymer strand or polymer ribbon, the preparation method may further comprise stitching and/or sewing the first soft tissue scaffold with the polymer strand or polymer ribbon to form a composite strand or ribbon.

Where the first collagen fiber bundle is a first dispersed collagen fiber bundle and the polymeric material is in the form of a polymer mesh, the preparation method may further comprise molding and/or layering the first dispersed collagen fiber bundle with the polymer mesh to form a composite scaffold.

The preparation method may further comprise stitching and/or sewing an additional soft tissue scaffold with the composite strand or ribbon to form a composite scaffold. The preparation method may further comprise weaving and/or knitting the composite strand or ribbon into a composite mesh, and molding and/or layering the composite mesh with an additional dispersed collagen fiber bundle to form a composite scaffold.

The present invention provides a preparation method of preparing a soft tissue composite, comprising: (a) dispersing a soft tissue to make dispersed collagen fiber bundles; (b) covering a top surface of a first mold with the dispersed collagen fiber bundles to form a first layer, wherein the first mold has a rim; (c) placing a polymer mesh on the top of the first layer to form the second layer, wherein the polymer mesh has an edge larger than the rim of the first mold; and (d) fixing the edge of the polymer mesh to the rim of the first mold, whereby a soft tissue composite is obtained. The preparation method may further comprise (e) placing a second mold on top of the polymer mesh, wherein the second mold has an open bottom, whereby the polymer mesh forms the bottom surface of the second mold; (f) covering a top surface of the second mold with the dispersed collagen fiber bundles to form a third layer; and (g) placing a lid to the top of the second mold. The preparation method may further comprise mixing dispersed collagen fiber bundles and polymer mesh by shaking or sonicating. The preparation method may further comprise freezing the soft tissue composite. The preparation method may further comprise dehydrating the frozen soft tissue composite. The polymer mesh may have been sterilized before the placing.

The present invention also provides a preparation method of preparing a soft tissue composite, comprising: (a) dispersing a soft tissue to make dispersed collagen fiber bundles; (b) covering the surface of a first mold with a polymer mesh; (c) transfer the dispersed collagen fiber bundles on top of the mesh; (d) inserting a second mold with a lid into the dispersed collagen fiber bundles; and (e) removing extra dispersed collagen fiber bundles from the rim of the second mold, whereby a soft tissue composite is obtained. The preparation method may further comprise mixing dispersed collagen fiber bundles and polymer mesh by shaking or sonicating. The preparation method may further comprise freezing the soft tissue composite. The preparation method may further comprise dehydrating the frozen soft tissue composite.

For each preparation method involving a polymer mesh, the preparation method may further comprise preparing the polymer mesh from a synthetic polymeric material in the form of a strand or ribbon using a process selected from the group consisting of: (a) knitting, warp-knitting, embroidering, crocheting or weaving the strand or ribbon; (b) injection molding; (c) 3D printing; and (d) electrospinning. The 3D printing may be carried out by fused deposition, selective sintering, or stereo lithography.

For each preparation method of the present invention, the soft tissue composite may have been terminally sterilized.

### BRIEF DESCRIPTION OF THE DRAWINGS

FIG. 1 shows a hemisphere-like, three-dimensional, large pore mesh scaffold with polygonal pores (1) produced from polymers (2).

FIG. 2 shows a hemisphere-like, three-dimensional, large pore mesh scaffold with a larger circular pore (3) at the apex, different sized polygonal pores (4, 5, 6) produced from polymers (7).

FIG. 3 shows a large pore mesh pattern made from different sized polygonal pores (8, 9) and polymers (10).

FIG. 4 shows a polygonal mesh scaffold with large pores (11) made from polymers (12).

FIG. 5 shows a dome-like, three-dimensional mesh scaffold showing the apex (13) at top and outer rim at bottom (17). The mesh has varying pore size and shape (14, 16) and is constructed from knitted polymer strands (15).

FIG. 6 shows a mesh scaffold pattern made from hexagonal pores (18) and fused (19) polymers (20).

FIG. 7 shows a mixed mesh pattern scaffold made from larger circular pores (21) and smaller, mixed geometry pores (22). The pattern has a combination of ribbons (23) and strands (24).

FIG. 8 shows a dome-like, three-dimensional, larger pore mesh scaffold made from non-resorbable polymeric strands that are bundled to create large pores and a thicker outer rim.

FIGS. 9A-G show a soft tissue scaffold made from dispersed collagen fiber bundles and non-resorbable polymer mesh (F-side view & G-top view). The machined mold that comprising parts 1-4 was designed and made (A) for this process, which includes placing the polymer mesh in the bottom mold (B), transferring dispersed collagen fiber bundles to the bottom mold on top of the mesh (C), placing the lid containing parts 2-4 on top of the bottom mold (D), and removing the excess dispersed collagen fiber bundle from the rim as shown in the side view (E). The entire assembly was frozen and freeze dried and the soft tissue composite was taken out (F & G).

FIGS. 10A-F show a pre-loaded soft tissue composite comprising dispersed collagen fiber bundles and resorbable polymer mesh pre-sutured with non-resorbable polymer sutures and fixed on a delivery device (A), including the top surface (joint facing, B) and bottom surface (tendon facing, C) of the composite. After being hydrated for less than 3 minutes (D and E), the delivery device was inserted through a passport cannula (F).

FIG. 11 shows a soft tissue composite made from dispersed collagen fiber bundles and polymer mesh and pre-sutured with polymer suture was handled with tweezers in a simulation testing chamber having a saline flow. During this process, the composite maintained structural integrity and surface smoothness, and avoided tissue detachment or delaminating from the embedded polymer mesh.

FIGS. 12A-B show a sandwich soft tissue composite comprising two layers of soft tissue scaffolds, a polymer mesh placed between the two layers of the soft tissue scaffolds (A), and optionally a polymer strand passing through the sandwich soft tissue composite (B).

FIGS. 13A-C show a top (A) or bottom (B) view of a soft tissue composite having one or more suture strands passing through the soft tissue composite as illustrated in FIG. 12B. The loose ends of the one or more suture strands are left at the four corners of the composite (C) or placed onto a delivery device for the easy delivery of soft tissue composite to a surgical site (A and B).

FIGS. 14A-B show a soft tissue composite comprising one layer of a soft tissue scaffold and a synthetic polymeric material and having one or more polymeric strands passing through the soft tissue scaffold in different stitch patterns.

FIGS. 15A-E show five stitch patterns for the soft tissue composite as illustrated in FIG. 14A.

FIGS. 16A-D show four stitch patterns for the soft tissue composite as illustrated in FIG. 14B.

FIGS. 17A-B show side views of two soft tissue composites comprising a synthetic polymeric material wrapping around a soft tissue scaffold (A) or a soft tissue scaffold is braided with two synthetic materials (B). The black arrows point to a soft tissue scaffold and white arrows point to the polymeric material.

FIGS. 18A-C show a soft tissue composite comprising one layer of a soft tissue scaffold and a polymeric material in the form of strands passing through the soft tissue scaffold in different stitch patterns (A-C).

FIGS. 19A-B show a soft tissue composite comprising one layer of a soft tissue scaffold and two polymer strands passing through the soft tissue scaffold and forming a stitching pattern on the top side (A) and another one on the bottom side (B).

FIGS. 20A-G show soft tissue composites made from split collagen fiber bundles (D and E) that were derived from a soft tissue (A-human fascia, B and C human tendon). The split collagen fiber bundles were braided with polymer strands to form a soft tissue composite (F and G).

FIG. 21 show a soft tissue scaffold being delivered to into a SterilCut press (BioCut Systems, Inc.).

FIG. 22A-C show a soft tissue composite strand made by coating a polymer strand with dispersed collagen fiber bundles (A and B), and collagen yarn made by removing the polymer strand from the soft tissue composite after lyophilization (C).

FIG. 23A-D show soft tissue composite in the form of a ribbon having a soft tissue scaffold sewed with polymeric strands (A and B) or the pressed soft tissue scaffold was encased in a ribbon made from non-resorbable polymers.

FIG. 24 show a polymer mesh made by 3D printing of one or more non-resorbable polymers in different designs.

FIG. 25A-D show the integrity of a sandwich soft tissue composite having two side layers of soft tissue scaffolds made of dispersed collagen fiber bundles and a polymer mesh buried as a middle layer between the two layers of the soft tissue scaffolds. After hydration and bending, the composite stayed intact and no separation of the layers was observed (A and B). After separation of the layers with hands, the dispersed collagen fiber bundles in one side layer were found interconnected with the polymer mesh and the dispersed collagen fiber bundles in the other side layer as indicated with black arrows (C and D).

FIG. 26A-C show a soft tissue scaffold before (A) and after being pressed (B and C). The pressed soft tissue scaffold was bent easily and showed much higher flexibility (C) as compared with an unpressed soft tissue scaffold.

FIG. 27A-B show an integrity test and measurement of force for a soft tissue composite passing through a cannular.

FIG. 28A-F show the 3D soft tissue composite used for breast implantation in a cadaver.

FIG. 29 show the soft tissue composite comprising dispersed collagen fiber bundles, soft tissue mesh, and sutures made from non-resorbable polymer under pulling.

FIGS. 30A-C show the hydrated soft tissue composite comprising dispersed collagen fiber bundle, mesh made from non-resorbable polymer, and sutures made from non-resorbable polymer passing through a canular (A), post canular pass (B), and reshaping after rehydration in saline (C).

FIGS. 31A-B show the illustration of the soft tissue composite comprising collagen fiber bundles wrapped around and interconnected with the polymer strand (A) and collagen fiber bundles passing through the pores of the polymer mesh and interconnected with the polymer mesh and the collagen fiber bundle from the other side (B).

FIGS. 32A-B show two delivery devices for a soft tissue composite: endoscopic clipper/applicator (A) and Trocar (B).

FIG. 33 shows a soft tissue composite with polymer mesh having hooks for fixation with adjacent soft tissue post implantation.

### DETAILED DESCRIPTION OF THE INVENTION

The present invention relates to a soft tissue composite for repairing a defective tissue. The soft tissue composite is biocompatible and exhibits desirable biomechanical strength. The soft tissue composite has a two-dimensional or three-dimensional shape. The invention is based on the surprising discovery by the inventors that a soft tissue composite comprising collagen fiber bundles, for example, a soft tissue scaffold, and a polymeric material, for example, a polymer mesh, which polymeric material may comprise a synthetic material, provides desirable biological and physical properties for repair of a defective tissue in a subject and added mechanical strength for implant delivery, handling during surgery, and supports biomechanical integrity after implantation. Such a soft tissue composite can be easily introduced to a defective tissue and provides a platform for attracting and differentiating stem cells. The invention also relates to a method of making the soft tissue composite and uses of the soft tissue composite in a medical device or tissue repair.

The inventors have developed soft tissue composites of different sizes and shapes with collagen fiber bundles of soft tissues isolated from human donors and polymeric materials interconnection with each other without using a carrier or chemical crosslinker. The inventors have discovered that a hydrated soft tissue composite can be compressed into different shapes and dimensions, and, upon rehydration in a liquid, the soft tissue composite can recover to its original shape and dimensions in a short time (e.g., less than 1 minute, 30 seconds, 15 seconds, 10 seconds, 5 seconds).

The term “soft tissue composite” as used herein refers to a combination of two, three or more materials, including a collagen fiber bundle of a soft tissue scaffold and a polymeric material, which may be a synthetic material, in, for example, layers. The layers in the soft tissue composite may be arranged in any way suitable for the intended uses. Each layer may comprise one or more materials. Each material may be native or synthetic.

The term “insoluble” as used herein refers to incapability of a substance to be dissolved in water.

Collagen is an insoluble protein commonly found in the extracellular matrix in soft tissues of animals, for example, connective tissues. A collagen molecule has a length of about 300 nm and a diameter of about 1.6 nm. In a soft tissue, the collagen molecules may form a collagen fibril having a diameter of about 100 nm and various length in micrometers, the collagen fibrils may form a collagen fiber having a diameter of about 10 μm and various length in millimeters, and the collagen fibers may form a collagen fiber bundle having a diameter of about 50 μm millimeters and various lengths in centimeters.

The term “collagen fiber” as used herein refers to an elongated structure in an extracellular matrix of a soft tissue, for example, a connective tissue. The collagen fiber is made up mainly of collagen fibril and collagen molecules. The collagen fiber may also comprise additional extracellular components that are naturally contained in the extracellular matrix of a soft tissue. The collagen fiber may have a length of about 10 mm-100 cm and a diameter of about 5-15 μm. The collagen fiber is typically arranged in branching bundles of indefinite length. In a preferred embodiment, the collagen fiber may be an insoluble fiber. It occurs in the skin, tendon, ligaments, bone, and cartilage.

The term “collagen fiber bundle” as used herein refers to a collection of collagen fibers in a soft tissue. The collagen fibers in the collagen fiber bundle may be connected as one piece. The soft tissue may be isolated from an animal including donation by a human. The collagen fiber bundle may have a length of about 10 mm-100 cm and a diameter of about 50 μm. The collagen fiber bundle used in or for making a soft tissue composite is insoluble. The collagen fiber bundle in the soft tissue composite may have a structure that is the same as or substantially identical to that naturally occurring in a soft tissue, which may be isolated from an animal including a human donor.

The collagen fiber bundle may be obtained by splitting of a soft tissue, and the obtained collagen fiber bundle is termed as “split collagen fiber bundle.” The split collagen fiber bundle is in a defined shape and may be dry or wet. The split collagen fiber bundle may have a structure that is the same or substantially identical to that of the collagen fiber bundle in a soft tissue, which may be isolated from an animal including a human donor. The split collagen fiber bundle is insoluble.

The collagen fiber bundle may be obtained by dispersing a soft tissue, and the obtained collagen fiber bundle is called as “dispersed collagen fiber bundle”. The dispersed collagen fiber bundle is in an amorphous form and typically wet. The dispersed collagen fiber bundle may have a structure that is the same or substantially identical to that of the collagen fiber bundle in a soft tissue, which may be isolated from an animal including a human donor. The split collagen fiber bundle is insoluble.

Soft tissue composite structure integrity is better than soft tissue or soft tissue scaffold alone because multi-phase entanglement between mesh structure and collagen fiber bundles during manufacturing processes. In one embodiment, polymer mesh containing macro and microscopic channels/pores made from 3D electrospinning may facilitate collagen fiber bundles entanglement to generate a multi-phase interconnected composite.

The term “polymer” is used herein to refer to a natural or synthetic substance composed of a large number of similar or same units bonded together. The units may also be called monomers or subunits.

The term “polymeric material” as used herein refers to a substance comprising one or more polymers. Each polymer may be natural or synthetic. The polymeric material used in or for making a soft tissue composite is insoluble. The polymeric material may be in any size, shape or form.

The term “scaffold” as used herein refers to a structure having a length, the longest dimension, no more than 10 times of its width, the second longest dimension.

The term “soft tissue (ST) scaffold” as used herein refers to a scaffold comprising one or more dispersed collagen fiber bundles. One or more dispersed collagen bundles may form a ST scaffold by, for example, molding. The ST scaffold may have a length, the longest dimension, no more than about 10 times of its width, the second longest dimension. The ST scaffold may comprise one or more other components.

The term “composite scaffold” as used herein refers to a scaffold comprising one or more collagen fiber bundles and one or more polymeric materials, or one or more soft tissue composites. One or more collagen fiber bundles and one or more polymeric materials, or one or more soft tissue composites may form a composite scaffold by, for example, molding, layering, stitching, and/or sewing. The composite scaffold may have a length, the longest dimension, no more than about 10 times of its width, the second longest dimension. The composite scaffold may comprise one or more other components.

The term “collagen yarn” as used herein refers to a structure comprising one or more dispersed collagen fiber bundles and having a length, the longest dimension, more than about 10 times of its width, the second longest dimension. One or more dispersed collagen fiber bundles may form a collagen yarn by, for example, braiding, pressing, twisting, weaving and/or knitting. The collagen yarn may comprise one or more other components.

The term “strand” as used herein refers to a structure having a length, the longest dimension, more than about 10 times of its width or diameter, the second longest dimension, and the width or diameter is not greater than about 1.5 mm. Two or more strands may be twisted around each other. The strand may be a suture.

The terms “polymer strand” and “polymer fiber” are used herein interchangeably and refer to a strand comprising one or more polymeric materials. One or more polymeric materials may form a polymer strand by for example, extrusion and/or spinning. The polymer strand may have a length, the longest dimension, more than about 10 times of its width or diameter, the second longest dimension, and the width or diameter is not greater than about 1.5 mm. The polymer strand may be a polymer suture. Two or more polymer strands may be twisted around each other.

The term “composite strand” as used herein refers to a strand comprising one or more collagen fiber bundles and one or more polymeric materials, or one or more soft tissue composites. One or more collagen fiber bundles and one or more polymeric materials, or one or more soft tissue composites may form a composite strand by, for example, braiding, coating, stitching and/or sewing. The composite strand may have a length, the longest dimension, more than about 10 times of its width or diameter, the second longest dimension, and the width or diameter is not greater than about 1.5 mm. The composite strand may be a composite suture.

The term “ribbon” as used herein refers to a structure having a length, the longest dimension, more than about 10 times of its width, the second longest dimension, and the width is greater than about 1.5 mm.

The term “polymer ribbon” as used herein refers to a ribbon comprising one or more polymeric materials. One or more polymeric material may form a polymer ribbon by, for example, printing, extrusion, spinning, and/or knitting, weaving, twisting or braiding from polymer strands. The polymer ribbon may have a length, the longest dimension, more than about 10 times of its width, the second longest dimension, and the width is greater than about 1.5 mm.

The term “composite ribbon” as used herein refers to a ribbon comprising one or more collagen fiber bundles and one or more polymeric materials, or one or more soft tissue composites. One or more collagen fiber bundles and one or more polymeric materials, or one or more soft tissue composites may form a composite ribbon by, for example, braiding, coating, stitching and/or sewing. The composite ribbon may have a length, the longest dimension, more than about 10 times of its width, the second longest dimension, and the width is greater than about 1.5 mm.

The term “mesh” as used herein refers to a structure formed by one or more materials and having openings such as holes, slits, cavities, voids, fenestrations, and channels. Such openings may extend from the surface to a part of the way or all of the way through of the mesh. The openings in the mesh may have different pore sizes, pore areas and/or void volumes.

The term “polymer mesh” as used herein refers to a mesh comprising one or more polymeric materials, polymer strands and/or polymer ribbons. One or more polymeric materials, polymer strands and/or polymer ribbons may form a polymer mesh by, for example, weaving and/or knitting, or additive manufacturing such as 3D printing and electrospinning.

The term “composite mesh” as used herein refers to a mesh comprising one or more collagen fiber bundles and one or more polymeric materials, or one or more soft tissue composites. One or more collagen fiber bundles and one or more polymeric materials, or one or more soft tissue composites may form a composite mesh by, for example, weaving and/or knitting, or additive manufacturing such as 3D printing and electrospinning. The term “mesh pattern” as used herein refers to a pattern or construction of a mesh in a soft tissue composite.

The term “stitching pattern” as used herein refers to a pattern of a strand or ribbon in a soft tissue composite.

The term “interconnect” as used herein refers to linking two substances via two or more connections. At each connection, the two interconnecting substances may be in touch with each other, or one substance may pass through the other substance.

The term “entangled with” as used herein refers to two interconnecting substances, one of which is wound or twisted around another or with another. The term “structural integrity” is used herein to refer to a state of being a whole and undivided structurally with respect to at least 80%, 85%, 90%, 95%, 99% or 100% of a soft tissue composite.

The term “surface smoothness” is used herein to refer to a surface lack of loosing fibers, which may be shown as waving fibers when fluid flows through the surface.

The term “crosslinker” as used herein refers to an agent capable of joining two or more molecules by a physical and/or chemical bond. A chemical bond may be an ionic, covalent, non-covalent, or metallic bond. Examples of a naturally occurring crosslinker include genipin (GNP), proanthocyanidin (PA), and epigallocatechin gallate (EGCG). A non-naturally occurring crosslinker may be selected from the group consisting of propylene glycol alginate, glycerol, sucrose octasulfate, polyethylene glycol, polymethylmethacrylate, polyurethane, acryloilmorpholine, N,N-dimethyl acrylamide, N-vinyl pyrrolidone and tetrahydrofurfuryl methacrylate, hydroxyapatite, polyurethane, and polylactic acid, glutaraldehyde, glyceraldehyde, poly(ethylene glycol) diepoxide crosslinker, poly(ethylene glycol) diglycidyl ether, EDC and NHS, transglutaminase, ethylenediamine, lysyl oxidase family, hexamethylene diisocyanate (HMDIC), dimethyl suberimidate (DMS), dimethyl-3-3′-dithiobispropionimidate (DTBP), and acryl azide, and/or combinations thereof. In additional embodiments, the dispersed soft tissue material described herein may optionally include a photoactive agent selected from the group consisting of a xanthene dye, naphthalimide compounds, riboflavin-5-phosphate, N-hydroxypyridine-2-(1H)-thione, N-(20-ethylaminoethyl)-4-amino-1,8-naphthalimide, bis-diazopyruvamide-N,N9-bis(3-diazopyruvoyl)-2,29-(ethylenedioxy) bis-(ethylamine) (DPD), diazopyruvoyl (DAP), methylene blue, erythrosin, phloxime, thionine, methylene green, rose Bengal, acridine orange, xanthine dye, thioxanthine dye, ethyl eosin, and eosin Y, and/or combinations thereof.

As indicated above, in one example, the making of a soft tissue scaffold or soft tissue composite from split collagen fiber bundles or dispersed collagen fiber bundles does not require, and thus in a preferred embodiment does not comprise, an additional crosslinker in addition to a natural crosslinker(s) from the soft tissue. In another embodiment, however, the making of a soft tissue scaffold or soft tissue composite from split collagen fiber bundles or dispersed collagen fiber bundles may optionally include an additional carrier in addition to a natural carrier(s) from the soft tissue. For example, the dispersed collagen fiber bundles in an alternative embodiment may optionally comprise, alginate, propylene glycol alginate, native or crosslinked chitosan, starch, polyethylene glycol, cellulose and its derivatives (such as cellulose acetate, carboxymethyl cellulose, and methyl cellulose), xanthan gum, dextran, hyaluronic acid, chondroitin sulfate, locust bean gum, gum tragacanth, gum arabic, curdlan, pullulan, scleroglucan, lower methoxylpectin, or carrageenan. The soft tissue composite may or may not optionally include a carrier solution. If included, the carrier solution may comprise salts of calcium, barium, aluminum, strontium, copper, zinc, magnesium, manganese, cobalt, or iron; glutaraldehyde, glyceraldehyde, genipin, glucose or ribose, poly(ethylene glycol) diepoxide crosslinker, poly(ethylene glycol) diglycidyl ether, EDC and NHS, transglutaminase, ethylenediamine, lysyl oxidase family, hexamethylene diisocyanate (HMDIC); dimethyl suberimidate (DMS), dimethyl-3-3′-dithiobispropionimidate (DTBP), or acryl azide. The optional carrier solution may also comprise natural and/or synthetic polymers such as native or modified collagen, gelatin, agarose, modified hyaluronic acid, fibrin, chitin, biotin, avidin, MATRIGEL®, HUMAN EXTRACELLULAR MATRIX®, proteoglycans, laminin, fibronectin, elastin, heparin, glycerol, polymethylmethacrylate, polyurethane, acryloilmorpholine, N,N-dimethyl acrylamide, N-vinyl pyrrolidone and tetrahydrofurfuryl methacrylate, hydroxyapatite, cross-linkage or functionalization of hyaluronan-based collagen and alginate, polyurethane, polylactic acid, or a combination comprising at least one of the foregoing polymers in addition to a natural carrier(s) from the soft tissue. In additional embodiments, for example, the soft tissue composite described herein may or may not include an optional additional carrier in addition to a natural carrier(s) from the soft tissue, wherein the carrier is selected from the group consisting of native collagen, hyaluronic acid, fibrin, chitin, biotin, avidin, MATRIGEL®, HUMAN EXTRACELLULAR MATRIX®, proteoglycans, laminin, fibronectin, elastin, heparin, alginate, genipin, chitosan, starch, glucose or ribose, cellulose and its derivatives (such as cellulose acetate, carboxymethyl cellulose, and methyl cellulose), xanthan gum, dextran, hyaluronic acid, chondroitin sulfate, locust bean gum, gum tragacanth, gum arabic, curdlan, pullulan, scleroglucan, lower methoxyl pectin, and carrageenan, and/or combinations thereof. Moreover, in further embodiments, the soft tissue composite described herein may or may not include an optional additional crosslinker in addition to a natural crosslinker(s) from the soft tissue, wherein the optional additional crosslinker is selected from the group consisting of alginate, starch, cellulose and its derivatives (such as cellulose acetate, carboxymethyl cellulose, and methyl cellulose), xanthan gum, dextran, carrageenan, genipin, hyaluronic acid, condroitin sulfate, locust bean gum, gum tragacanth, gum arabic, curdlan, pullulan, scleroglucan, and lower methoxylpectin, glucose or ribose, native collagen, hyaluronic acid, fibrin, chitin, biotin, avidin, MATRIGEL®, HUMAN EXTRACELLULAR MATRIX®, proteoglycans, laminin, fibronectin, elastin, heparin, and chitosan, and/or combinations thereof.

The term “subject” as used herein refers to a human individual having a defective tissue. The subject is in need of defective tissue repair. The subject may be a patient. The subject may be a human donor of a soft tissue used to make a collagen fiber bundler in a soft tissue composite.

The term “biocompatible” as used herein refers to a material that does not provoke an adverse response in a patient. For example, a suitable biocompatible soft tissue composite when introduced into a subject does not by itself provoke a significant immune response, and is not toxic to the subject.

The term “biomechanical strength” as used herein refers to properties exhibited by a soft tissue, soft tissue scaffold, mesh or soft tissue composite, including flexibility rigidity, drape coefficient, suture retention strength, tensile strength, ball-burst strength, stiffness, tear resistance, and absorption time in vivo.

The term “flexibility rigidity” as used herein refers to key characteristics for material handling. Flexibility is the ability of material being bended without breaking. Rigidity is defined as the property exhibited by the solid to change in its shape.

The term “drape coefficient” as used herein refers to a ratio of the projected draped specimen area divided by the projected undraped specimen area.

The term “suture retention strength” as used herein refers to a peak strength when the suture is pulled out the wall of the sutured material. It is related to the difficulty of the suturing operation and the firmness of the suture connection, and it is an important biomechanical property.

The term “tensile strength” also called “ultimate tensile strength (UTS)” as used herein refers to the maximum stress a material can withstand while being stretched or pulled before breaking.

The term “ball-burst strength” as used herein refers to a measurement of the resistance to mechanical penetration of a material such as tissue products.

The term “stiffness” as used herein refers to the extent to which a material resists deformation in response to an applied force such as stretching and pulling. This can be measured as modulus of elasticity or Young's modulus in tension as the slope on the initial linear elastic region of the stress/strain curve.

The term “tear resistance” as used herein refers to a measurement of how well a material can withstand the effects of tearing.

The term “absorption in vivo” as used herein refers to degradation or removal of a mesh from a subject by hydrolysis, assimilation of a mesh through diffusion or osmosis in a subject, and/or intake or endocytosis of a mesh by cells in the body of a subject.

The term “absorption time in vivo” as used herein refers to time taken for absorption of a mesh in a subject. At the end of absorption, the mesh may not be detectable through regular histological analysis, such as paraffin embedding, sectioning, hematoxylin and eosin staining, and imaging under light microscope.

The term “porous tissue material” as used herein refers to a three-dimensional tissue structure that is porous, elastic, flexible, fibrous, and resilient. In addition, the preferred “porous tissue material” is substantially unitary, coherent, and/or cohesive in the sense of holding together in a single piece and/or staying substantially intact. As used herein, the terms “coherent” or “cohesive” refer to the property that the elements of the tissue structure are maintained substantially intact (in the sense of holding together in a unitary structure rather than becoming disassembled or separated). In a dry state, the porous tissue material may quickly absorb fluid. In the wet state, the porous tissue material may maintain the porosity, cohesiveness, and/or integrity. The wet porous structure may resist certain external stress or strain, and bounce back and reabsorb fluid after being released from external stress or strain.

The term “processed tissue material” as used herein refers to a native, normal tissue that has been procured from an animal source (e.g. human or non-human, such as bovine, porcine, canine including, but not limited to, a dog, equine, ovine, or non-human primate including, but not limited to, ape and gorilla, in origin), preferably a mammal, and mechanically cleaned of attendant tissues and/or chemically cleaned of cells and cellular debris.

The term “two-dimensional structure” as used herein refers to a structure of a soft tissue composite, soft tissue scaffold or mesh whose cross section has a thickness that is no more than 10%, 9%, 8%, 7%, 6%, 5%, 4%, 3%, 2%, 1%, 0.5%, 0.25%, 0.1%, 0.001%, 0.0001% or 0.00001% of the average diameter of the cross section.

The term “three-dimensional structure” as used herein refers to a structure of a soft tissue composite, soft tissue scaffold or mesh whose cross section has a thickness that is at least 10%, 20%, 30%, 40%, 50%, 60%, 70%, 80%, 90%, 100%, 500% or 1,000% of the average diameter of the cross section.

The term “thickness” as used herein refers to the shortest dimension of a structure for example, of a soft tissue composite, soft tissue scaffold or mesh.

The term “length” as used herein refers to the longest dimension of a structure, for example, of a soft tissue composite, soft tissue scaffold or mesh.

The term “width” as used herein refers to the second longest dimension of a structure of, for example, of a soft tissue composite, soft tissue scaffold or mesh.

The term “diameter” as used herein refers to the diameter of a cross section of a structure of, for example, of a soft tissue composite, soft tissue scaffold or mesh.

The terms “projection” and “height” are used herein interchangeably and refer to a three-dimensional depth of a structure, for example, of a soft tissue composite, soft tissue scaffold or mesh.

The term “plasticizer” as used herein refers to any biocompatible compound that can easily displace/replace water at the molecular level and preferably has a low molecular weight such that the plasticizer fits into the spaces available to water within the molecular structure of the bone or soft tissue. Such plasticizers are preferably not toxic to the cellular elements of tissue into which the tissue is to be placed.

The soft tissue composite may be used for wound repair, dental procedures, shoulder and/or rotator cuff repair and reconstruction, tendon or ligament repair and/or reconstruction, sports medicine procedures, hernia treatment procedures, joint repair and/or reconstruction, minimally invasive surgeries, orthopedic surgeries, and/or in plastic or cosmetic procedures in a subject. The soft tissue composite may also be used as a carrier for cells, biological factors such as platelet rich plasma, bone marrow aspirate, and growth factors, or a host tissue.

The soft tissue composite may have a size, shape, structure and thickness suitable for a predetermined procedure, thereby eliminating the need for substantial processing or cutting of the soft tissue prior to implantation. Suitable shapes for the soft tissue composite include curved or straight edges. Suitable shapes may include circular, elliptical, rectangular, cylinder, square, triangular, elongated, or other shapes. The soft tissue composite may be shaped as regular or irregular polygons.

The invention provides a soft tissue composite. The soft tissue composite comprises a first collagen fiber bundle of a first soft tissue and a polymeric material. The first collagen fiber bundle interconnects with the polymeric material. The first soft tissue may be isolated from a first animal or human donor.

The polymeric material may be entangled with the first collagen fiber bundle. The polymeric material may comprise pores, and the first collagen fiber bundle passes through the pores. The polymeric material may be a mesh, strand, or ribbon.

The soft tissue composite may lack a chemical crosslinker. In one embodiment, the first collagen fiber bundle and the polymeric material are not crosslinked by a non-naturally occurring crosslinker, for example, a chemical compound.

The soft tissue composite may lack a carrier, for example, a polymeric or collagen carrier. In one embodiment, the soft tissue composite excludes a non-naturally occurring carrier, for example, a polymeric or collagen carrier.

The first collagen fiber bundle may not have a surface treatment. The surface treatment may include surface modification by heating, freezing, plasticizing, and/or light treatment such as ultraviolet light treatment. Post-op side-ness effect that provides one side of soft tissue composite with soft tissue for tissue integration/healing, and the other side of polymeric mesh for adhesion prevention features; these features are desired for abdominal wall conformability.

The first soft tissue may be selected from the group consisting of muscle, fat, blood vessel, nerve, tendon, ligament, lining of joints, skin, dermis, pericardium, fascia, cartilage, dura mata, endocardium, mucosal tissue, placental membrane, periosteum, bladder, small intestine, large intestine, urethra, and placenta.

The first collagen fiber bundle may have been frozen, freeze-dried, dehydrated and/or plasticized.

The first collagen fiber bundle may be selected from the group consisting of first split collagen fiber bundle, first dispersed collagen fiber bundle and a combination thereof. The first collagen fiber bundle may be a first split collagen fiber bundle prepared by splitting a soft tissue. The first collagen fiber bundle may be a first dispersed collagen fiber bundle prepared by dispersing a soft tissue. The soft tissue may be isolated from an animal or human donor.

The first dispersed collagen fiber bundle may be in the form of a first soft tissue scaffold or a first collagen yarn. The first dispersed collagen fiber bundle may be molded to form a first soft tissue scaffold. The first dispersed collagen fiber bundle may be twisted, pressed, rolled, and/or spun to form a first collagen yarn.

The polymeric material may be selected from the group consisting of a synthetic polymeric material, a natural polymeric material, and a combination thereof. The polymeric material may comprise a synthetic polymeric material, a natural polymeric material, or a combination thereof.

The polymeric material may be in the form of a polymer strand, a polymer ribbon or a polymer mesh. The polymeric material may be extruded and/or spun to form a polymer strand. The polymeric material may be extruded and/or spun to form a polymer ribbon. The polymeric material may be woven, knitted, crocheted and/or printed to form a polymer mesh.

The polymeric material may comprise one or more biodegradable or resorbable polymers. The one or more biodegradable polymers may be selected from the group consisting of polylactic acid (PLA), polyglycolide/polyglycolic acid (PGA), polyglycolide/lactide (PGLA), poly-4-hydroxybutyrate, polydioxanone (PDO), poly-p-dioxanone, trimethylene carbonate, polyglycolide/caprolactone (PC-Cl), polycaprolactone (PCL), silk, polyhydroxyalkanoate (PHA), 3-hydroxybutyrate (3HB), 4-hydroxybutyrate (4HB), polyglycolide-trimethylene carbonate (PG-TMC). and a combination thereof. The synthetic material may comprise one or more non-absorbable polymers selected from the group consisting of polypropylene (PP), polyester (PET), poly(ethylene terephthalate) (PET) and poly(butylene terephthalate) (PBT), polyamide (Nylon-6 and Nylon-6,6), Nylon, polyethylene, ultra-high molecular weight polyethylene (UHMWPE), silk fibroin, p-Gomore silk worm, and a combination thereof. The one or more biodegradable polymers may be selected from the group consisting of polylactic acid, polyglycolic acid, poly-4-hydroxybutyrate, polydioxanone (PDO), trimethylene carbonate, polycaprolactone, silk, silk fibroin, polyhydroxyalkanoate (PHA), 3-hydroxybutyrate (3HB), 4-hydroxybutyrate (4HB), and a combination thereof.

The first collagen fiber bundle may be a first split collagen fiber bundle. The polymeric material may be in the form of a polymer strand or a polymer ribbon. The split first collagen fiber bundle and the polymer strand or polymer ribbon may form a composite strand or ribbon by, for example, braiding. The composite strand may be a composite suture. The composite suture may be formed by the composite strand by braiding or knitting.

The first collagen fiber bundle may be a first dispersed collagen fiber bundle. The polymeric material may be in the form of a polymer strand or polymer ribbon. The first dispersed collagen fiber bundle and the polymer strand or polymer ribbon may form a composite strand or ribbon by, for example, coating, wrapping, and/or braiding. The first dispersed collagen fiber bundle may be in the form of a first collagen yarn.

The first collagen fiber bundle may be first dispersed collagen fiber bundle. The polymeric material may be in the form of a polymer mesh. The first dispersed collagen fiber bundle and the polymer mesh may form a composite scaffold by, for example, molding and/or layering.

The first collagen fiber bundle may be in the first isolated soft tissue. The polymeric material may be in the form of a polymer strand or polymer ribbon. The first isolated soft tissue and the polymer strand or polymer ribbon may form a composite strand or ribbon or a composite scaffold by, for example, stitching and/or sewing.

The first collagen fiber bundle may be a first dispersed collagen fiber bundle in the form of a first soft tissue scaffold. The polymeric material may be in the form of a polymer strand or polymer ribbon. The first soft tissue scaffold and the polymer strand or polymer ribbon may form a composite strand or ribbon or a composite scaffold by, for example, stitching and/or sewing.

The composite strand or ribbon may further comprise an additional collagen fiber bundle. The additional collagen fiber bundle may be from the same soft tissue as the first collagen fiber bundle. The additional collagen fiber bundle may be an additional dispersed collagen fiber bundle in the form of an additional soft tissue scaffold by, for example, molding. The additional soft tissue scaffold and the composite strand or ribbon may form a composite scaffold by, for example, stitching and/or sewing.

The composite strand or ribbon may further comprise an additional collagen fiber bundle. The additional collagen fiber bundle may be from the same soft tissue as the first collagen fiber bundle. The composite strand or ribbon may form a composite mesh by, for example, weaving and/or knitting. The additional dispersed collagen fiber bundle and the composite mesh may form a composite scaffold by, for example, molding and/or layering.

The soft tissue composite may comprise a first layer, and the first layer may comprise the first collagen fiber bundle. The first collagen fiber bundle may be a first dispersed collagen fiber bundle in the form of a first soft tissue scaffold, and the polymeric material may be a synthetic material. The soft tissue scaffold may be pressed. The term “pressed” and “compressed” are used herein interchangeably. The pressed soft tissue scaffold may have a thickness to less than 50% of the unpressed soft tissue scaffold. The soft tissue scaffold may be dry and flexible. The dry soft tissue scaffold may be bend to less than 150° without breaking.

The synthetic material may pass through the first soft tissue scaffold. The synthetic material may be a polymer strand, for example, suture. The polymer strand may have various stitch patterns.

The first soft tissue scaffold and the synthetic material may form a woven pattern, for example, a braided or wrapping pattern (FIGS. 17A-B). A weaving pattern may be formed by two or more materials in a complicated or skilled way, and the materials may be in the form of a strand. A braided pattern may be a type of a woven pattern, and made up of three or more interlaced strands. A wrapping pattern may be a way of covering or enclosing an inner layer with an outer layer.

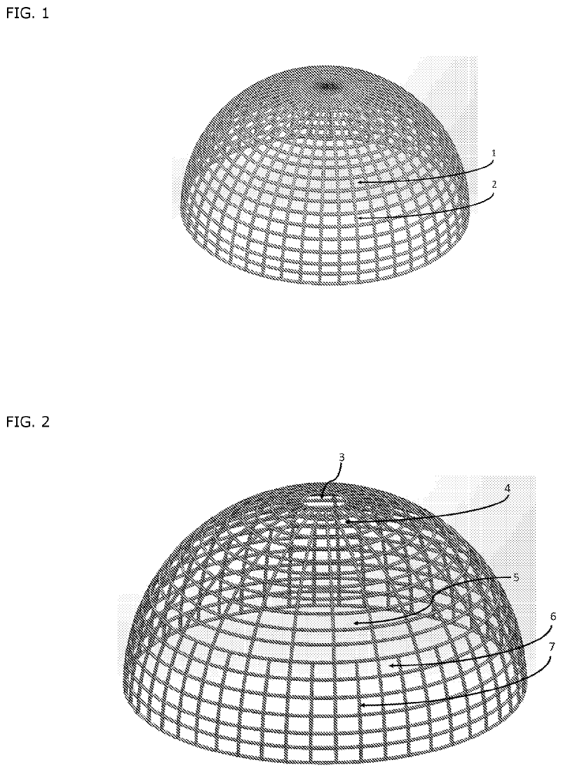

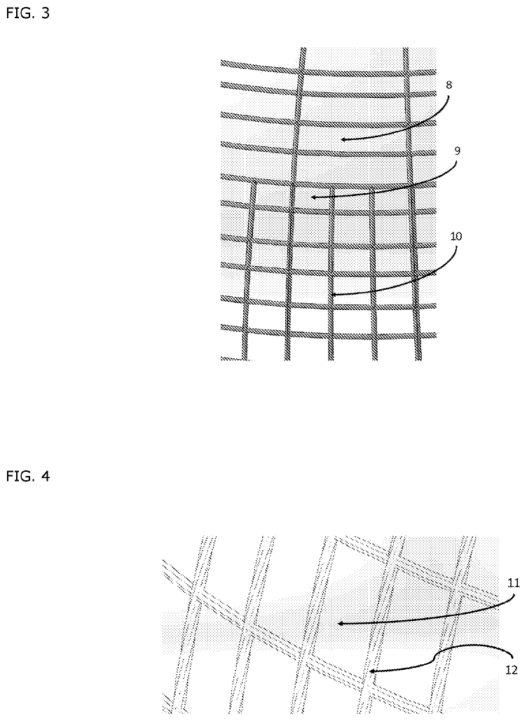

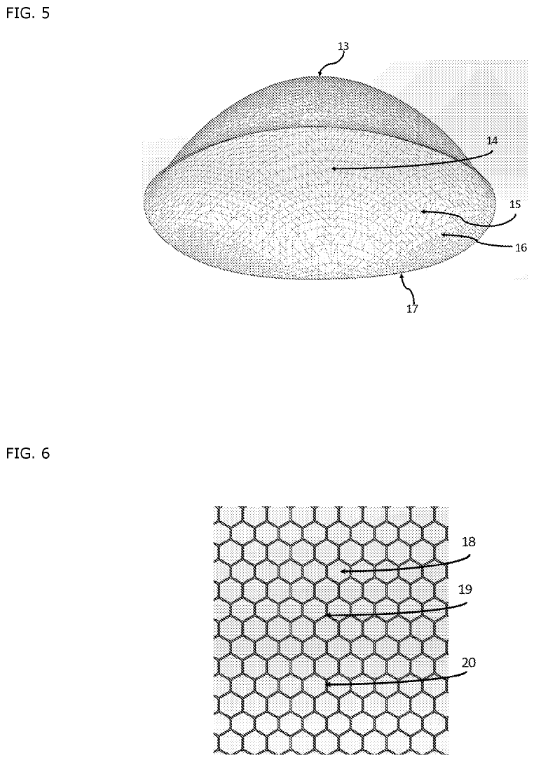

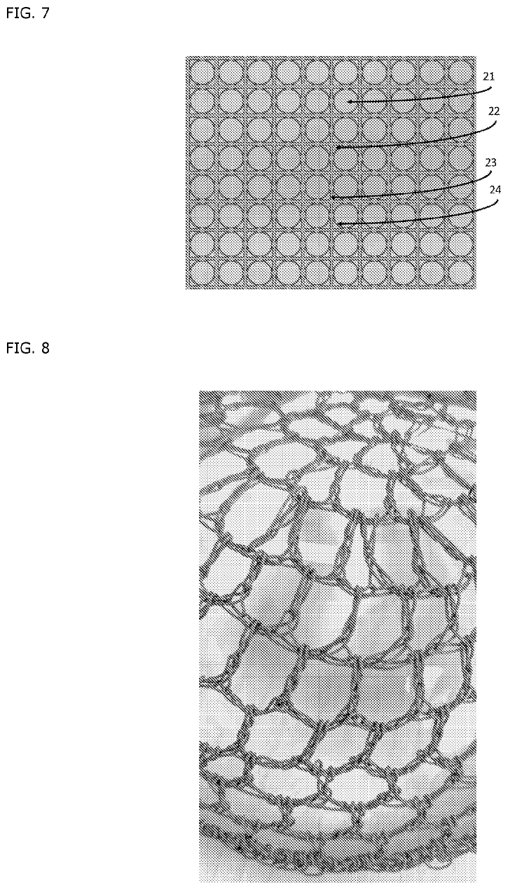

The soft tissue composite may further comprise a second layer. The second layer may comprise the polymeric material in the form of a polymer mesh. The polymeric material may have different mesh patterns (FIGS. 1-8, 24).

The first layer and the second layer may be arranged in any way. The two layers may be in contact with each other, directly or indirectly. The one layer may be on top of the other layer. The two layers may overlap with each other, completely or partially. One layer may wrap around the other layer. One layer may cover the other layer. For example, the first layer may cover the second layer. The first layer may wrap around the second layer, completely or partially.

The second layer may further comprise a natural polymeric material. The natural polymeric material may be a natural material. Examples of the natural material include collagen, silk fibroin, fibronectin, laminin, cellulose and proteoglycans.

The first layer may cover about 5-100%, 20-95% or 40-90% of the surface of the second layer.

The soft tissue composite may have any size, shape, structure, or thickness suitable for achieving an intended use of the soft tissue composite. For example, a soft tissue composite has a size, shape, structure, and/or thickness that match with the size, shape, structure, and/or thickness of a tissue defect, which the soft tissue composite is intended to repair. The physical, biological and/or chemical properties of the mesh may be modified so that the soft tissue composite would exhibit the desirable features.

The polymer mesh may have a thickness of 0.01-10 mm, preferably 0.1-2 mm

The polymer mesh may have a projection or height of 2-20 cm, preferably 5-15 cm.

The polymer mesh may have a thickness no less than 0.01 mm and a width or diameter of 7-30 cm, preferably 14-20 cm.

The polymer mesh may have a thickness no greater than 5 mm and a width or diameter of 0.1-100 cm, 0.1-50 cm, 0.1-10 cm, 0.1-3 cm, 0.1-2 cm, 0.1-1 cm, 7-20 cm, or 14-19 cm.

The polymer mesh may have a thickness no greater than 5 mm and a length of 1-500 cm, 1-400 cm, 1-300 cm, 1-200 cm, 1-100 cm, 1-50 cm, 1-20 cm, 7-20 cm, or 14-19 cm.

The polymer mesh may have an average pore size from 2 mm to 20 mm, or from 5 mm to 12 mm. The polymer mesh may have an average of pore area from 4 mm² to 400 mm², or from 25 mm² to 144 mm². The polymer mesh may have different patterns to provide different advantages. For example, the polymer mesh may have a pattern such that breaking one side of the boundary of the polymer mesh would not have any impact on the pores adjacent to the boundary and the strength of the polymer mesh. The polymer mesh may have different pore shapes, e.g. a circular, elliptical or polygonal shape or a combination thereof. The polymer mesh may be constructed such that cutting or trimming of the polymer mesh would not result in fraying or unraveling of the polymer mesh beyond the trimmed or cut portion and would not compromise mechanical strength of the surrounding fibers or fiber bundles. In some cases, the pore size may vary at different places of the same polymer mesh. Some areas of the polymer mesh may have bigger pores to enable stronger integration of soft tissue scaffolds from the top and bottom layers when the polymer mesh is located between the top and bottom layers of soft tissue scaffolds.

The polymer mesh may have a flexibility rigidity of 1-200 μNm, 2-115 μNm, or 2-50 μNm. The first soft tissue scaffold may have a drape coefficient of 50-5%. The polymer mesh may have a suture retention strength of each polymer mesh fiber, fiber bundle or strut in the range of 5-500 N, 5-400 N, 5-300 N, or 5-200 N. The polymer mesh may have a tear resistance of 5-200 N, 5-100 N, or 5-50 N. The soft tissue composite may have a tear resistance of 5-200 N, 5-100 N, or 5-50 N. The soft tissue composite may have a tensile strength of 20-200 N/cm, 50-150 N/cm, or 50-100 N/cm. The soft tissue composite may have a ball-burst strength of 20-200 N, 50-150 N, or 50-100 N. The soft tissue composite may have a stiffness of 10-800 N/m, 10-500 N/m, or 20-300 N/cm.

The polymer mesh may have an absorption time in vivo from 30 days to 18 months, from 60 days to 12 months, or from 60 days to 6 months.

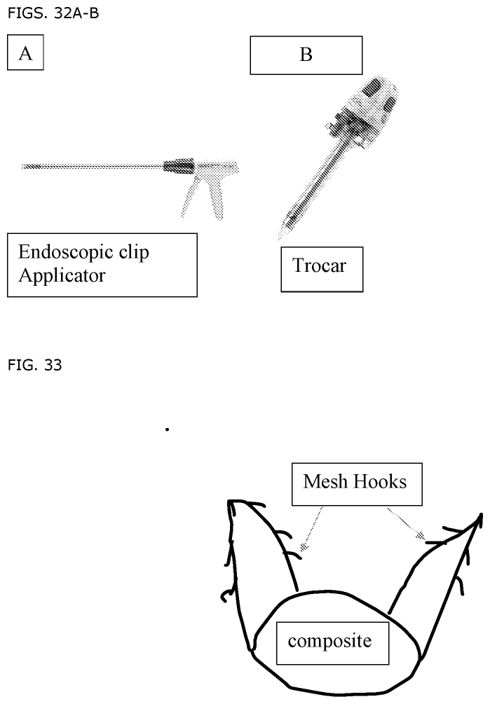

In another embodiment, polymer mesh may have hooks made to facilitate the host tissue fixation with soft tissue composite without the need of staplers or extensive suturing. This may potentially reduce the post-operational pain and facilitate the peripheral fixation. The peripheral fixation enables the mesh to conform to the abdominal wall during the tissue integration process. Suture mesh with hooks (FIG. 32) extended outside and peripheral of the soft tissue composite may be used to facilitate self-attachment to the host tissue.

The polymer mesh made by co-extruding two types of polymers such as of non-resorbable polymer and resorbable polymer forming a strand or ribbon in the mesh with non-resorbable core and resorbable surface to provide novel properties such as short-term tissue integration (resorbable surface going away for tissue in-growth) and mid/long-term strength durability (non-resorbable polymer stability). In another embodiment, the core may be another resorbable polymer with long resorption time such as 2 years or longer. The surface of strands or ribbons can also be hydrophilic or hydrophobic pending the resorption time needs.

In the soft tissue composite, the polymer mesh may maintain at least 50%, 60%, 70%, 80%, 85%, 90%, 95% or 100% of its mechanical strength for at least 30 days in vivo, at least 3 months, or at least 6 months. The soft tissue composite may maintain at least 50%, 60%, 70%, 80%, 85%, 90%, 95% or 100% of its mechanical strength for at least 30 days, at least 3 months, or preferably at least 6 months in vivo.

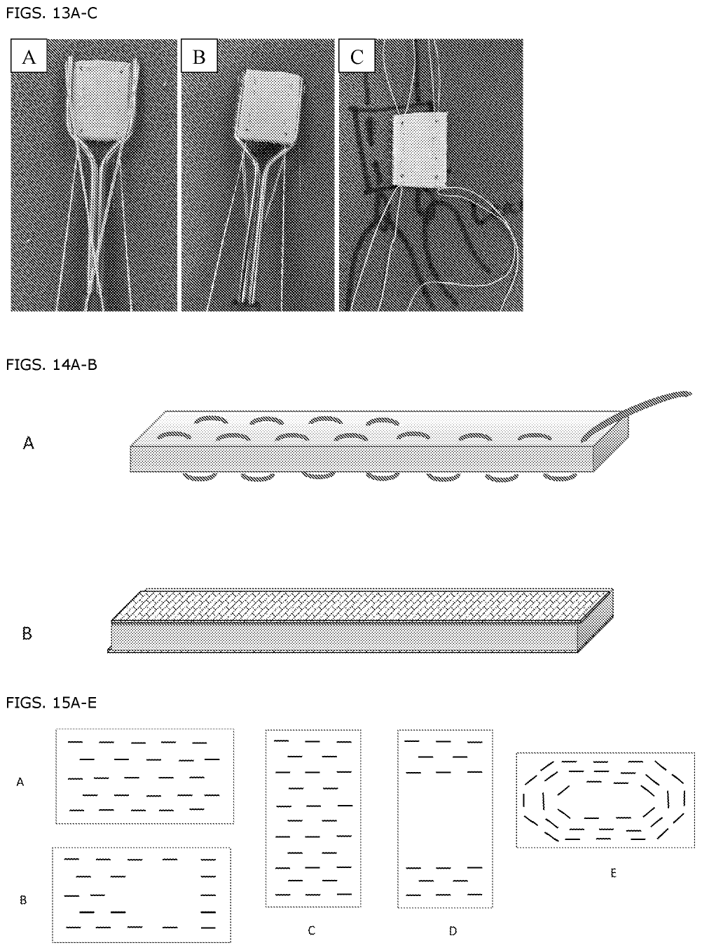

In addition to a first layer comprising a first collagen fiber bundle and a second layer comprising a polymeric material, the soft tissue composite may further comprise a third layer. The resulting soft tissue composite is referred to a sandwich soft tissue composite. The second layer may be between the first layer and the third layer. The third layer may comprise a third collagen fiber bundle of a third soft tissue, which may be isolated from the first human donor or another human donor. The third collagen fiber bundle may be in the form of a third soft tissue scaffold. As a result, the mesh may be placed between two layers of soft tissue scaffolds (FIGS. 12A-B and 13A-D). The soft tissue composite may be pre-sutured (FIGS. 12B and 13A-D). Such a soft tissue composite is also referred to as a sandwich soft tissue composite. The first soft tissue scaffold and third soft tissue scaffold may be different. The first soft tissue scaffold and third soft tissue scaffold may be identical.

The first soft tissue and the third soft tissue may be different. The first soft tissue and the third soft tissue may be identical and may be isolated from the same human donor or different human donors.

In the sandwich soft tissue composite, the polymeric material may be a polymer mesh. The polymer mesh may have pores, and the first collagen fiber bundle from a first soft tissue scaffold and the third collagen fiber bundle from a third soft tissue scaffold interconnect with each other through the pores. The polymeric material may be entangled with the third collagen fiber bundle. The first collagen fiber bundle from a first soft tissue scaffold and the third collagen fiber bundle from a third soft tissue scaffold may be different. The first collagen fiber bundle from a first soft tissue scaffold and the third collagen fiber bundle from a third soft tissue scaffold may be from the same soft tissue preparation.

The third soft tissue may be selected from the group consisting of muscle, fat, blood vessel, nerve, tendon, ligament, lining of joints, skin, dermis, pericardium, fascia, cartilage, dura mata, endocardium, mucosal tissue placental membrane, periosteum, bladder, small intestine, large intestine, urethra, and placenta.

The second soft tissue scaffold may comprise a second dispersed soft tissue material. The first dispersed soft tissue material and the second dispersed soft tissue material may be different. The first dispersed soft tissue material and the second dispersed soft tissue material may be identical.

The third collagen fiber bundle may have been frozen, freeze-dried, and/or plasticized. The third collagen fiber bundle may lack a non-naturally occurring crosslinker, a non-naturally occurring carrier or a combination thereof.

In the sandwich soft tissue composite, the second layer may further comprise a second collagen fiber bundle of a second soft tissue, which may be isolated from a second animal or human donor. The second collagen fiber bundle may be a second dispersed collagen fiber bundle. The second dispersed collagen fiber bundle may cover the synthetic material.

In another aspect, the soft tissue may be dispersed at a temperature above about −80, −70, −60, −50, −40, −30, −20, −10, −5, 0, 5, 10, 15, 16, 17, 18, 19, 20, 21, 22, 23, 24, 25, 26, 27, 28, 29, 30, 31, 32, 33, 34, 35, 36 or 37° C. In some embodiments, the soft tissue may be dispersed at a temperature below about −70, −60, −50, −40, −30, −20, −10, −5, 0, 5, 10, 15, 16, 17, 18, 19, 20, 21, 22, 23, 24, 25, 26, 27, 28, 29, 30, 31, 32, 34, 35, 36, 37, 38 or 39° C. In additional embodiments, the soft tissue may be dispersed at a temperature of about −80, −70, −60, −50, −40, −30, −20, −10, −5, 0, 5, 10, 15, 16, 17, 18, 19, 20, 21, 22, 23, 24, 25, 26, 27, 28, 29, 30, 31, 32, 33, 34, 35, 36, or 37° C. In further embodiments, the soft tissue may be dispersed at a temperature between about −80 and about 50° C., −20 and about 50° C., −10 and about 50° C., about −5 and about 50° C., about 0 and about 50° C., about 0 and about 37° C., about 5 and about 24° C., about 10 and about 24° C., about 15 and about 24° C., about −5 and about 10° C., about −5 and about 15° C., or about 0 and about 15° C. In another aspect, the soft tissue may be dispersed mechanically by chopping, skiving, milling, grinding, slicing and/or beating the soft tissue (e.g., by a blender, a beater, and a mixer). In some embodiments, the temperature of the soft tissue may rise above ambient temperature due to the dispersing process, but no additional heat is applied to the soft tissue. In a preferred embodiment, the temperature of soft tissue may be controlled by adding, e.g., cold solution (e.g., water and saline) or ice (e.g., made from water or isotonic solution) to the soft tissue prior to, or during, the dispersing process. In another embodiment, the method may exclude treating the soft tissue with heat above about 24, 25, 26, 27, 28, 29, 30, 31, 32, 33, 35, 36, 37, 38, 40, 45, 50, 60, 70, 80, 90, 100, 150, or 200° C. prior to, during and/or after the dispersing. In some embodiments, the method excludes treating the soft tissue with heat below about 50, 70, 90, or 110° C. prior to, during, and/or after the dispersing. In other embodiments, the method excludes treating the soft tissue with heat between about 26 and about 200° C., about 30 and about 150° C., about 40 and about 120° C., about 50 and about 110° C., and about 50 and about 100° C. prior to, during, and/or after the dispersing. In another aspect, the method may exclude sonication, microwave irradiation, or conventional heat transfer from a heating component, among other methods known in the art.

In some embodiments, the soft tissue may be dispersed for about 5 seconds, 10 seconds, 15 seconds, 20 seconds, 25 seconds, 30 seconds, 40 seconds, 50 seconds, 60 seconds, 2 minutes, 3 minutes, 4 minutes, 5 minutes, 10 minutes, 15 minutes, 30 minutes, 60 minutes, 2 hours, 5 hours, 10 hours, 24 hours, 48 hours or 72 hours or more. In additional embodiments, the soft tissue may be dispersed for about 20 seconds, 30 seconds, 60 seconds, 2 minutes, 3 minutes, 4 minutes, 5 minutes, 10 minutes, 15 minutes, 30 minutes, 60 minutes, 2 hours, 5 hours, 10 hours, 24 hours, 48 hours or 72 hours or less. In further embodiments, the soft tissue may be dispersed for between about 20 seconds and 72 hours, about 30 seconds and 30 minutes, about 30 seconds and 20 minutes, about 30 seconds and 10 minutes, preferably between about 1 minute and 20 minutes, between about 1 minute and 10 minutes, or between about 1 minute and 6 minutes.

In some embodiments, the soft tissue is dispersed in the presence of a solution or solvent (e.g., water, and saline solution). The solution or solvent may be in the form of liquid or solid. In additional embodiments, the soft tissue may be dispersed in the presence of a solid (e.g. ice formed from water, and solid formed from saline solution). The solid may comprise one or more salt granulate and/or one or more sugar granulate. The salt granulate may comprise NaCl₂ and/or CaCl₂), and the sugar granulate may comprise glucose, sucrose, and/or fructose. In another embodiment, the solid may have a size of about 0.5 mm³, 1 mm³, 2 mm³, 4 mm³, 10 mm³, 2 cm³, 4 cm³, 6 cm³, 8 cm³, 10 cm³, or above. In additional embodiments, the size of a solid may be between about 0.5 mm³ and 20 cm³, between about 1 mm³ and 20 mm³, between about 5 mm³ and 20 cm³, between about 1 cm³ and 20 cm³, or between about 1 cm³ and 10 cm³. In further embodiments, the method described herein may further comprise dissolving the solid during and/or after dispersing the soft tissue. In yet additional embodiments, a weight ratio of a moist soft tissue to a solution or solvent in the dispersed collagen fiber bundles (in other words, the wet weight of tissue to the solution) is about 0.01, 0.05, 0.1, 0.2, 0.3, 0.4, 0.5, 0.6, 0.7, 0.8, 0.9 or 1.0 or more. The weight ratio of the dispersed collagen fiber bundles to solution or solvent in the dispersed collagen fiber bundles may also be about 0.02, 0.05, 0.1, 0.2, 0.3, 0.4, 0.5, 0.6, 0.7, 0.8, 0.9 or 1.0 or less. Further, the weight ratio of the soft tissue to the solution or solvent in the dispersed collagen fiber bundles may be from about 0.010 to 10.0, preferably from about 0.02 to 2.0, more preferably from about 0.04 to 1.0, from about 0.05 to 1.5, from about 0.05 to 1.0, or from about 0.1 to 1.0.

In some embodiments, the weight percentage of the soft tissue in the dispersed collagen fiber bundles is about 2, 5, 10, 15, 20, 30, 40, 50, 60, 70, 80, 90, 92, 94, 96, 98, 100% or more in a dry state. In additional embodiments, the weight percentage of the soft tissue in the dispersed collagen fiber bundles is about 3, 5, 10, 15, 20, 30, 40, 50, 60, 70, 80, 90, 91, 93, 95, 97, 99, 100% or less in a dry state. In further embodiments, the weight percentage of the soft tissue in the dispersed collagen fiber bundles is from about 2% to about 100%, preferably from about 50% to about 90%, from about 50% to about 80%, from 60% to 100%, from 80% to about 100%, or from about 60% to about 100% in a dry state.

In some embodiments, the weight percentage of the dispersed collagen fiber bundles in the soft tissue scaffold or the soft tissue composite is about 50, 60, 70, 80, or 90% or more in a dry state. In additional embodiments, the weight percentage of the dispersed collagen fiber bundles in the soft tissue scaffold or the soft tissue composite is about 50, 60, 70, 80, 90, or 100% or less in a dry state. In further embodiments, the weight percentage of the dispersed collagen fiber bundles in the soft tissue scaffold or the soft tissue composite is from about 50% to about 100%, from about 70% to about 100%, from about 80% to about 100%, or from about 90% to about 100% in a dry state. The amount of the dispersed collagen fiber bundles in the soft tissue scaffold or the soft tissue composite may be varied to adjust the density, porosity, and/or viscosity characteristics of the soft tissue scaffold or the soft tissue composite as well as the re-hydration characteristics of the resulting porous structure. Moreover, incorporating an additional dispersed collagen fiber bundles in the soft tissue scaffold or the soft tissue composite may strengthen the three-dimensional framework and increase the integrity of the resulting porous structure. Incorporating more dispersed collagen fiber bundles in the soft tissue scaffold or the soft tissue composite also may decrease or increase the cellular response towards the framework of the resulting porous structure by facilitating cellular attachment, migration, and/or proliferation.

In some embodiments, the dispersed collagen fiber bundles may be resistant to absorption after implantation. For example, the soft tissue scaffold or soft tissue composite formed from the dispersed collagen fiber bundles may maintain 30-100% of its thickness from the first month of implantation through 6 months after implantation; maintain 50-100% of its thickness from the first month of implantation through 6 months after implantation; maintain 50-80% of its thickness from the first month of implantation through 6 months after implantation; maintain 30-100% of their thickness from the first month of implantation through 12 months after implantation; or maintain 50-80% of their thickness from the 1st month of implantation through 12 months after implantation.

In one example, the dispersed collagen fiber bundles does not require, and thus in a preferred embodiment does not comprise, an additional crosslinker or carrier in addition to natural (i.e., endogenous) crosslinker(s) and natural carrier(s) from the one or more soft tissue(s). Thus, in a preferred embodiment, the methods and resulting products may consist essentially of (and/or consist of) natural crosslinker(s) and natural carrier(s) from the one or more soft tissue(s). In another embodiment, however, the methods and resulting products may optionally include the addition of additional crosslinker(s) or carrier(s) in addition to the natural crosslinker(s) and natural carrier(s) from the one or more soft tissue(s) after dispersing the soft tissue, and, accordingly, the soft tissue scaffold and/or soft tissue composite may optionally comprise such additional non-natural crosslinker(s) or carrier(s).

The soft tissue composite may be dry. The soft tissue composite may be hydrated, for example, before use. The hydrated soft tissue composite may be pressed by force. The pressed hydrated soft tissue composite may have a thickness less than 50% of its original thickness at a dry state. The pressed and hydrated soft tissue composite may be capable of regaining the original shape and thickness after the pressing force is removed. The soft tissue composite may be surface treated.

In the sandwich soft tissue composite, the first layer and the second layer may be stitched together. The first layer, the second layer and the third layer may be stitched together.

The methods described herein may further comprise freezing, drying, or freeze-drying the dispersed collagen fiber bundles to produce a soft tissue scaffold and/or soft tissue composite. In another embodiment, the freezing and freeze-drying may be conducted at a controlled freezing/cooling rate. The controlled freezing/cooling rate may be from about 1° C. to 20° C. per minute, from about 2° C. to 10° C. per minute, from about 3° C. to 10° C. per minute, or from about 3° C. to 6° C. per minute. In some embodiments, the soft tissue scaffold and/or soft tissue composite may be freeze-dried to a point such that the freeze-dried fragments have an average residual moisture of less than about 10, 5, 4, 3, 2, 1, 0.5, or 0.1 wt %. In further embodiments, the soft tissue scaffold and/or soft tissue composite may be dried, and/or freeze-dried to a point such that the dried or freeze-dried fragments have residual moisture from about 0.01% to 10%, from about 0.01% to 5%, from about 0.01% to 3%, from about 0.1% to 3%, from 0.5% to 3%, or from 1% to 3%.

In some embodiments, the soft tissue scaffold and/or soft tissue composite may be desiccated. For example, the freeze-dried soft tissue scaffold and/or soft tissue composite may be desiccated in an oven, a desiccator, or in package(s) with desiccant (in packets or other forms), and maintained at a temperature from 10° C. to 100° C., from 15° C. to 60° C., from 20° C. to 55° C., or from 20° C. to 40° C. for a period of time from 7 days to 5 years, from 14 days to 120 days, or from 14 days to 70 days.

In another aspect, the dispersed collagen fiber bundles consists essentially of and/or consists of the one or more soft tissue(s); and solution or solvent. In some embodiments, the soft tissue scaffold consists essentially of and/or consists of components from the one or more soft tissue(s). The term “essentially consisting of” defines the scope of the soft tissue scaffold and/or soft tissue composite to include additional elements that do not materially affect the porosity or void fraction of the soft tissue scaffold and/or soft tissue composite consisting of initial elements. For example, the dispersed soft tissue material consisting essentially of one or more soft tissue(s) may include elements in addition to the one or more soft tissue(s) that do not materially affect the porosity or void fraction of the dispersed soft tissue material consisting of the one or more soft tissue(s). Materially affecting the porosity or void fraction herein means changing the porosity or void fraction at least by about 0.5, 1, 2, 3, 4, 5, 7, 9, 10, 12, 15, 20, 25, 30, or 40%.

In some embodiments, the soft tissue scaffold or soft tissue composite may comprise pores having an average or mode diameter of about 1, 5, 10, 100, 200, 300, 400, 500, 700, 1000, 1500, 2000, 3000, or 4000 μm or more. In additional embodiments, the soft tissue scaffold or soft tissue composite may comprise pores having an average or mode diameter of about 2, 6, 20, 100, 200, 300, 400, 500, 700, 900, 1000, 1300, 1500, 2000, 3000, or 4000 μm or less. In further embodiments, the soft tissue scaffold or soft tissue composite may comprise pores having an average or mode diameter from about 1 μm to 4000 μm, from 1 μm to 1000 μm, from about 10 μm to 1000 μm, from about 20 μm to 500 μm, from about 20 μm to 350 μm, from about 50 μm to 350 μm on an average. In some embodiments, the soft tissue scaffold or soft tissue composite may have up to 70% of pores with a diameter less than 50 μm. In some embodiments, the soft tissue scaffold or soft tissue composite may have more than 30% pores with a diameter from about 50 μm to 350 μm. In some embodiments, the soft tissue scaffold or soft tissue composite may comprise more than 50% pores with a diameter from about 20 μm to 350 μm.

In some embodiments, an average void volume of the soft tissue scaffold or soft tissue composite may be about 10, 20, 30, 40, 50, 60, 70, 80, 90, 95, 96, 97, 98, 99% or more. In additional embodiments, an average void volume of the soft tissue scaffold or soft tissue composite may be about 10, 20, 30, 40, 50, 60, 70, 80, 90, 95, 96, 97, 98, 99% or less. In further embodiments, an average void volume of the soft tissue scaffold or soft tissue composite may be from about 10% to about 99%, from about 30% to 99%, from about 50% to about 99%, from about 70% to 99%, from about 80% to about 99%, or from about 80% to 96%. In some embodiments, the average void volume of the soft tissue scaffold or soft tissue composite may be from about 1 cc/to 30 cc/g, from about 2 cc/g to 20 cc/g, from about 3 cc/g to 20 cc/g, from about 5 cc/g to 20 cc/g. In some embodiments, the average void volume of the soft tissue scaffold may be controlled by adjusting the volume of liquid added to the dispersed soft tissue material prior to drying or freeze drying.

In some embodiments, the soft tissue scaffold or soft tissue composite may be compressible after hydration and re-formable after weight removal. The shape and dimension of the soft tissue scaffold may elastically return to original shape and dimension after substantial compression, e.g., with 50 grams to 1000 grams, with 100 grams to 1000 grams, or with 100 grams to 500 grams; or less than 2000 grams, less than 1000 grams, or less than 500 grams. The time required to return to original shape and dimension after substantial compression may be less than 30 seconds, less than 20 seconds, less than 10 seconds, or less than 5 seconds. The dimension change of the soft tissue scaffold may be less than 80%, less than 70%, less than 60%, less than 50%, less than 40%, less than 30%, less than 20%, or less than 10% after the weight removal.

In some embodiments, the soft tissue scaffold or soft tissue composite may be pressed before hydration to obtain a thinner soft tissue scaffold or soft tissue composite. The pressing force may be from 1 ton to 60 tons, from 5 tons to 60 tons, from 5 tons to 55 tons, from 10 tons to 50 tons, from 10 tons to 40 tons, or from 15 tons to 35 tons. The pressed thickness may be reduced relative to the uncompressed thickness by from 10% to 95%, from 10% to 85%, from 20% to 80%, from 30% to 80%, from 40% to 90%, from 50% to 90%. The soft tissue scaffold or soft tissue composite may be configured to retain its compressed shape while in a dehydrated state. The shape and dimension of the soft tissue scaffold or soft tissue composite may be configured to return to original shape and dimension after hydration.

In some embodiments, the soft tissue scaffold or soft tissue composite prepared by the methods described herein may have collagen fiber, collagen fiber bundle dimensions or diameters more similar to its natural state, compared to some other processing techniques in the prior art. In some embodiments, the fibers or fiber bundles in the soft tissue scaffold are intertwined or randomly interwoven. Previous techniques have led to solubilized collagen or a collagen fiber with smaller sizes than the natural collagen fibers or collagen fiber bundles and thus may degrade faster in vivo. Moreover, the soft tissue in the methods described herein is preferably dispersed without being denatured, micronized, or cryofractured, thus preferably having no change or only minimal change of the extracellular matrix macromolecule components (for example: collagen, proteoglycan, elastin, hyaluronic acid, laminin, fibronectin, among other), and having no change or only minimal change in the relative ratio of macromolecule components in the dispersed collagen fiber bundle, and/or the soft tissue scaffold and/or soft tissue composite. In the other words, the extracellular matrix macromolecules of the resulting soft tissue scaffold or soft tissue composite are preferably not modified (or at least not substantially modified). Dispersing the soft tissue according to the disclosed methods may open the structure of the soft tissue to facilitate cell infiltration and/or tissue-in-growth after implantation, but preferably may not modify the soft tissue scaffold and/or soft tissue composite interaction at the micro scale level, unlike implants prepared by other techniques. At the same time, the fiber and/or fiber bundle dimension (e.g. diameter, or width, and length) of a preferred soft tissue scaffold and/or soft tissue composite may support a framework with opened pore structure and with a network of fibers and/or fiber bundles that may provide a relatively strong and stable framework without needing additional (non-natural) crosslinking or adding a carrier. In some embodiments, the fiber or fiber bundle dimension may provide a stable framework for the soft tissue scaffold and/or soft tissue composite without modifying or weakening the integrity and cohesiveness of the soft tissue scaffold and/or soft tissue composite. In preferred embodiments, the structure of the soft tissue scaffold and/or soft tissue composite can stay intact after rehydration and agitation in liquid, and the soft tissue scaffold and/or soft tissue composite can allow for biocompatible cellular and tissue response and good volume retention after implantation in an animal. The volume retention after implantation in an animal may be measured by the largest cross-section area of the implanted soft tissue composite at different times after implantation. For example, the soft tissue scaffold and/or soft tissue composite may have recipient's cell infiltration and angiogenesis after 1-4 weeks of implantation, and maintain the soft tissue scaffold and/or soft tissue composite volume (e.g. the largest cross-section area of implanted soft tissue scaffold and/or soft tissue composite) from about 30% to 100% between 4 week and 24 weeks of implantation, from about 40% to 100% between 4 week and 24 weeks of implantation, or from about 50% to 100% between 4 week and 24 weeks of implantation.

In some embodiments, the soft tissue scaffold and/or soft tissue composite may comprise fibers or fiber bundles having an average diameter of about 0.1, 0.5, 1, 3, 5, 10, 20, 30, 40, 50, 60, 70, 80, 90, 100, 110, 120, 130, 140, 150, 200, 250, 300, 350, 400, 450, 500 μm or more. In additional embodiments, the soft tissue scaffold and/or soft tissue composite may comprise fibers or fiber bundles having an average diameter of about 0.1, 0.5, 1, 3, 5, 10, 20, 30, 40, 50, 60, 70, 80, 90, 100, 110, 120, 130, 140, 150, 200, 250, 300, 350, 400, 450, 500 μm or less. In further embodiments, the soft tissue scaffold and/or soft tissue composite may comprise fibers or fiber bundles having an average diameter from about 0.1 μm to about 500 μm, from about 0.1 μm to about 200 μm, from about 1 μm to about 500 μm, from about 10 μm to about 500 μm, from about 100 μm to about 1000 μm, or from about 100 μm to about 500 μm.

In some embodiments, the soft tissue scaffold and/or soft tissue composite comprises fibers and/or fiber bundles having an average length of 5 μm, 10 μm, 50 μm, 100 μm, 1000 μm, 5000 μm, 1 cm, 2 cm, 5 cm, 10 cm, 12 cm, 15 cm, 20 cm, 25 cm, or 50 cm or more. In some embodiments, the soft tissue scaffold and/or soft tissue composite comprises fibers and/or fiber bundles having an average length of 5 μm, 10 μm, 50 μm, 100 μm, 1000 μm, 5000 μm, 1 cm, 2 cm, 5 cm, 10 cm, 12 cm, 15 cm, 20 cm, 25 cm, or 50 cm or less. In additional embodiments, the soft tissue scaffold and/or soft tissue composite comprises fibers and/or fiber bundles having an average length from about 5 μm to about 50 cm, from about 100 μm to about 50 cm, from about 1000 μm to about 50 cm, from about 1 cm to about 50 cm, from about 1 cm to about 30 cm, from about 1 cm to about 20 cm, or from about 1 cm to about 15 cm.

The polymeric material may comprise a first synthetic polymer, a second synthetic polymer, and a third synthetic polymer. The first synthetic agent, the second synthetic agent, and the third synthetic agent may be identical. The first synthetic agent, the second synthetic agent, and the third synthetic agent may be different. The second synthetic agent may be connected to the first synthetic agent.

The soft tissue composite may further comprise a suture. The surgical suture may have been pre-positioned on the soft tissue scaffold or soft tissue composite to save the surgical procedure time. An exemplary surgery is a rotator cuff repair procedure in a shoulder joint.

The suture may be synthetic. The pattern of the I suture may vary depending on the design of the delivery device. The suture may be in continuous mattress pattern or interrupted suture pattern. The suture may run through the soft tissue scaffold or soft tissue composite. The suture may run in parallel with any one side of the soft tissue composite. The suture may have long ends left in the soft tissue composite and used for pre-loading the soft tissue composite onto a delivery device.

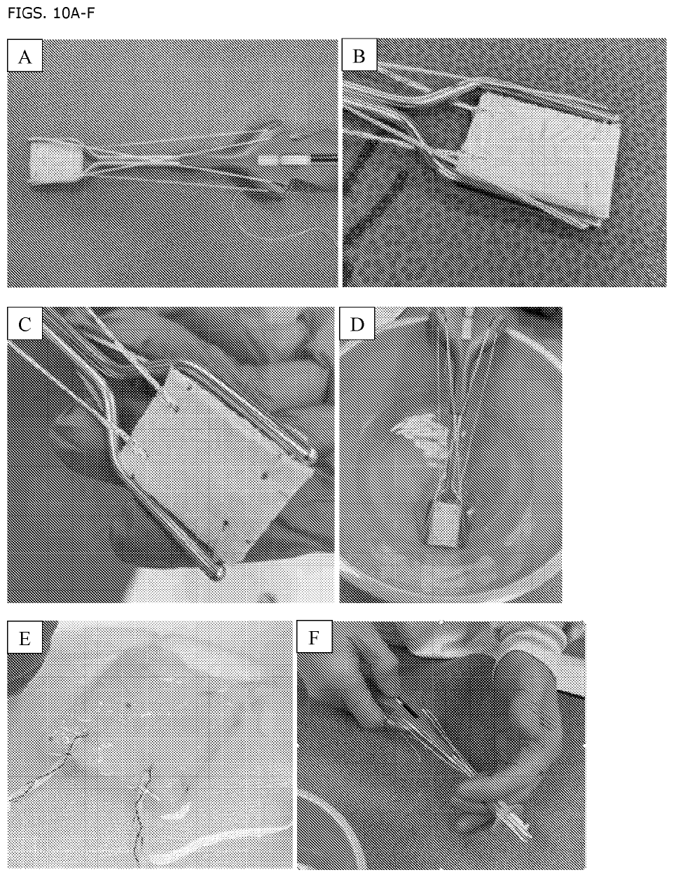

The soft tissue composite may be an implant, which may be referred to as a composite implant or composite graft. The soft tissue composite implant may be pre-loaded onto a delivery device for implantation or delivery into a surgical implantation site (FIG. 10). The soft tissue composite may have a top surface, for example, facing a joint, four sides, and a bottom surface, for example, facing a tendon (FIG. 10). The top surface and bottom surface may have same or different pore sizes. In the soft tissue composite, the mesh may be between two layers of soft tissue scaffolds. The soft tissue composite may have been dehydrated and sterilized.

The pre-sutured and pre-loaded composite graft may be hydrated, then inserted through a portal or cannula to be inserted into the joint. The composite graft may then be positioned onto a surgical site and fixed with suture anchor(s), sutures, or staples.

The soft tissue composite may be treated with UV irradiation, a heating process, E-beam or gamma irradiation. The resulting soft tissue composite may have an improved structural integrity. The treatment may be performed prior to a drying process, during the dry phase, or after dehydration.

In one embodiment, the top surface of a soft tissue composite may face to the joint cavity and have a smaller pore size while the bottom face may contact a bone or soft tissue and have a bigger pore size. The surface with a smaller pore size may be treated with heat, for example, by contacting a heated surface. This heating treatment may be performed prior to or after the soft tissue composite is dehydrated.

In another embodiment, a dehydrated soft tissue composite may be exposed to a heated surface to generate a smooth composite surface. This treatment may improve maintenance of structure integrity and surface smoothness of the soft tissue composite after the soft tissue composite is exposed to a liquid flow or other manipulations during an arthroscopic surgery. This heating treatment may be applied to any one or more of the surfaces, for example, top, bottom and side surfaces, of the soft tissue composite. In one embodiment, the heating treatment may be applied only to the soft tissue composite surface facing or exposed to a joint side. The soft tissue composite surface facing a tendon or bone may be porous to enhance tissue ingrowth and integration. The heat treatment may last between 5 seconds and 5 minutes, 10 seconds and 4 minutes, 20 seconds and 3 minutes, 30 seconds and 3 minutes, or 30 seconds and 2 minutes. The temperature of the heated surface may be between 40° C. and 400° C., 50° C. and 300° C., 50° C. and 200° C., or 100° C. and 250° C.

In another embodiment, a dehydrated soft tissue composite may be exposed to a UV light to generate a smooth composite surface. This treatment may improve maintenance of structure integrity and surface smoothness of the soft tissue composite after the soft tissue composite is exposed to a liquid flow or other manipulations during an arthroscopic surgery. This UV light treatment may be applied to any one or more of the surfaces, for example, top, bottom and side surfaces, of the soft tissue composite. In one embodiment, the UV light treatment may be applied only to the soft tissue composite surface facing or exposed to a joint side. The soft tissue composite surface facing a tendon or bone may be porous to enhance tissue ingrowth and integration. The UV light treatment may last between 30 seconds and 30 minutes, 1 minute and 30 minutes, 5 minutes and 25 minutes, 5 minutes and 20 minutes, or 5 minutes and 15 minutes. The intensity generated from UV light box may be between 100 mJ/cm\hat{ }2 and 1000 mJ/cm\hat{ }2, 200 mJ/cm\hat{ }2 and 1000 mJ/cm\hat{ }2, 300 mJ/cm\hat{ }2 and 1000 mJ/cm\hat{ }2, or 400 mJ/cm\hat{ }2 and 800 mJ/cm\hat{ }2.

For each soft tissue composite according to the present invention, a medical device comprising the soft tissue composite is provided. The mesh may have pores, and the first soft tissue scaffold and the second soft tissue scaffold may interconnect with each other in the pores such that the soft tissue composite is stabilized. The medical device may further comprise a breast implant, pacemaker implant, microchip implant, metal bone implant, a drug delivery device implant, internal monitor implant, allograft tissue implantation, autograft tissue implantation, device implant, bolster flap transfer, vascular graft, stent, or a combination thereof.

The present invention also provides a method of repairing a defective tissue in a subject in need thereof. The repair method comprises administering the soft tissue composite or the medical device of the present invention to the defective tissue in the subject.

During Intra-op, ease of use, better structure integrity and compatibility of soft tissue composite with current arthroscopic, laparoscopic and endoscopic procedure using trocar, applicators (see FIG. 32) are desirable features include reduced pain due to smaller incisions, reduced hemorrhaging, and shorter recovery time.

Polymer mesh conforms to the abdominal cavity and mesh with hooks Porous soft tissue with large pores (for example 1.5 mm×1.8 mm) for consistent tissue integration. Hydrophilic polymeric mesh to prevent and minimize undesired tissue adhesion post-surgery.

The method may further comprise suturing the soft tissue composite to the defective tissue. The soft tissue composite or the medical device is administered to the subject via a delivery device. The delivery device may a syringe, forceps, tubing, catheter, endoscopic clip or any other graft holder.

Where the soft tissue composite is dry, the repair method may further comprise hydrating the dry soft tissue composite before the administering.

Where the soft tissue composite has a surface, the repair method may further comprise placing the smoother surface towards to the lumen or joint space and the opposite surface towards to repairing tissue.

According to the repair method, the defective tissue may be a joint surface lesion (JSL) in an articular cartilage or subchondral bone.

The present invention further provides a method of making the soft tissue composite of the present invention. The preparation method comprises interconnecting the first collagen fiber bundle with the polymeric material. The interconnecting lacks chemical crosslinking.

The first collagen fiber bundle may be a first split collagen fiber bundle, and the method may further comprise splitting the first soft tissue isolated from the first donor to make the first split collagen fiber bundle.

The first collagen fiber bundle may be a first dispersed collagen fiber bundle, and the method may further comprise dispersing the first soft tissue to make the first dispersed collagen fiber bundle. The first dispersed collagen fiber bundle may be in the form of a first soft tissue scaffold, and the method may further comprise molding the first dispersed collagen fiber bundle into the first soft tissue scaffold. The first dispersed collagen fiber bundle may be in the form of a first collagen yarn, and the method may further comprise twisting, spinning, rolling, and/or pressing the first dispersed collagen fiber bundle into the first collagen yarn.

The polymeric material may be in the form of a polymer strand or polymer ribbon, and the method may further comprise extruding and/or spinning the polymeric material into the polymer strand or polymer ribbon.

The polymeric material may be in the form of a polymer mesh, and the method may further comprise printing such as 3D printing the polymeric material into a polymer mesh.

The first collagen fiber bundle may be a first split collagen fiber bundle, the polymeric material may be in the form of a polymer strand or polymer ribbon, and the method may further comprise braiding the first split collagen fiber bundle with the polymer strand or polymer ribbon to form a composite strand or ribbon.

The first collagen fiber bundle may be a first dispersed collagen fiber bundle, the polymeric material may be in the form of a polymer strand or polymer ribbon, and the method may further comprise coating and/or braiding the first dispersed collagen fiber bundle with the polymer strand or polymer ribbon to form a composite strand or ribbon. The first dispersed collagen fiber bundle may be in the form of a collagen yarn.

The first collagen fiber bundle may be in the first soft tissue, the polymeric material may be in the form of a polymer strand or polymer ribbon, and the method may further comprise stitching and/or sewing the first soft tissue with the polymer strand or polymer ribbon to form a composite strand or ribbon.

The first collagen fiber bundle may be in the form of a first soft tissue scaffold, the polymeric material may be in the form of a polymer strand or polymer ribbon, and the method may further comprise stitching and/or sewing the first soft tissue scaffold with the polymer strand or polymer ribbon to form a composite strand or ribbon.

The first collagen fiber bundle may be a first dispersed collagen fiber bundle, the polymeric material may be in the form of a polymer mesh, and the method may further comprise molding and/or layering the first dispersed collagen fiber bundle with the polymer mesh to form a composite scaffold.

The method may further comprise stitching and/or sewing an additional soft tissue scaffold with the composite strand or ribbon to form a composite scaffold. The additional soft tissue scaffold may be the first dispersed collagen fiber bundle in the form of a soft tissue scaffold.

The method may further comprise weaving and/or knitting the composite strand or ribbon into a composite mesh, and molding and/or layering the composite mesh with an additional dispersed collagen fiber bundle to form a composite scaffold.

In one embodiment, a method of preparing a soft tissue composite is provided. The method comprises (a) dispersing a soft tissue to make dispersed collagen fiber bundles; (b) covering a top surface of a first mold with the dispersed collagen fiber bundles to form a first layer, wherein the first mold has a rim; (c) placing a polymer mesh on the top of the first layer to form the second layer, wherein the polymer mesh has an edge larger than the rim of the first mold; and (d) fixing the edge of the polymer mesh to the rim of the first mold, whereby a soft tissue composite is obtained. The method may further comprise (e) placing a second mold on top of the polymer mesh, wherein the second mold has an open bottom, whereby the polymer mesh forms the bottom surface of the second mold; (f) covering a top surface of the second mold with the dispersed collagen fiber bundles to form a third layer; and (g) sealing the second mold. The method may further comprise mixing dispersed collagen fiber bundles and polymer mesh by shaking or sonicating prior to freezing. The method may further comprise freezing the soft tissue composite. The method may further comprise dehydrating the frozen soft tissue composite. The polymer mesh may have been sterilized before the placing. The soft tissue composite may have been terminally sterilized.

In another embodiment, a method of preparing a soft tissue composite is provided. The method comprises (a) dispersing a soft tissue to make dispersed collagen fiber bundles; (b) covering the surface of a first mold with a polymer mesh; (c) transfer the dispersed collagen fiber bundles on top of the mesh; (d) inserting a second mold with a lid into the dispersed collagen fiber bundles; and (e) Removing extra dispersed collagen fiber bundles from the rim of the second mold, whereby a soft tissue composite is obtained. The method may further comprise mixing dispersed collagen fiber bundles and polymer mesh by shaking or sonicating prior to freezing. The method may further comprise freezing the soft tissue composite. The method may further comprise dehydrating the frozen soft tissue composite. The soft tissue composite may have been terminally sterilized.

The preparation method may further comprise freezing the soft tissue composite. The preparation method may further comprise dehydrating the frozen soft tissue composite.

According to the preparation method of the present invention, the polymer mesh may be prepared by subjecting the synthetic material in the form of a strand or ribbon to a process. The process may comprise knitting, warp-knitting, embroidering, crocheting or weaving the polymer strand or ribbon, whereby a mesh is formed; knitting, warp-knitting, embroidering, crocheting or weaving the polymer strand or ribbon, whereby knots and/or intersections of strands or ribbons are formed and fused to each other by, for example, melting or softening; injection molding; 3D printing; and electrospinning. The 3D printing may be carried out by fused deposition, selective sintering, or stereo lithography. The mesh may be packaged before the placing. The mesh may be sterilized before the placing.

According to the preparation method of the present invention, a soft tissue scaffold may be prepared by placing a dispersed collagen fiber bundle in a mold having a predetermined shape, wherein the dispersed collagen fiber bundle is frozen, dehydrated such as freeze-drying, and/or plasticized in the mold.

The preparation method may further comprise storing the soft tissue scaffold and/or soft tissue composite prior to implanting. In some embodiments, the soft tissue scaffold and/or soft tissue composite may be stored in a dry state, in cryopreservation, or in a wet state. In additional embodiments, the preparation method may further comprise treating the soft tissue scaffold and/or soft tissue composite with a water replacing agent. In further embodiments, the soft tissue scaffold and/or soft tissue composite may be stored in a wet state. In yet further embodiments, the water replacing agent comprises one or more selected from the group consisting of glycerol (glycerin USP), adonitol, sorbitol, ribitol, galactitol, D-galactose, 1,3-dihydroxypropanol, ethylene glycol, triethylene glycol, propylene glycol, glucose, sucrose, mannitol, xylitol, meso-erythritol, adipic acid, proline, hydroxyproline, polyethylene glycol, alcohol, and lipids. In another aspect, the method described herein may further comprise plasticizing the soft tissue scaffold and/or soft tissue composite.

The preparation method may further comprise treating the soft tissue scaffold and/or soft tissue composite with one or more treatment solutions before or after freezing drying, and/or freeze drying (or before or after other methods for drying the soft tissue scaffold, besides freeze drying, such as air dry or drying in a drying oven at a pre-set temperature). The preparation method may further comprise treating the soft tissue scaffold and/or soft tissue composite with one or more treatment solutions after freezing, drying, and/or freeze drying before implantation. In some embodiments, the treatment solution comprises an ionic, enzymatic, or chemical crosslinking agent, a photoactive agent, or a polymer. The ionic crosslinking agent may comprise one or more selected from the group consisting of calcium, barium, aluminum, strontium, copper, zinc, magnesium, manganese, cobalt, and iron. The enzymatic crosslinking agent may comprise one or more selected from the group consisting of transglutaminase, ethylenediamine, lysyl oxidase family, hexamethylene diisocyanate (HMDIC), dimethyl suberimidate (DMS), and dimethyl-3-3′-dithiobispropionimidate (DTBP). The chemical crosslinking agent comprises one or more selected from the group consisting of glutaraldehyde, glyceraldehyde, genipin, glucose or ribose, poly(ethylene glycol) diepoxide crosslinker, poly(ethylene glycol) diglycidyl ether, EDC and NHS, and acryl azide. The polymer may comprise one or more selected from the group consisting of native or modified collagen, gelatin, agarose, modified hyaluronic acid, fibrin, chitin, biotin, avidin, demineralized bone matrix, MATRIGEL®, HUMAN EXTRACELLULAR MATRIX®, proteoglycans, laminin, fibronectin, elastin, heparin, glycerol, sucrose octasulfate, polyethylene glycol, polymethylmethacrylate, polyurethane, acryloilmorpholine, N,N-dimethyl acrylamide, N-vinyl pyrrolidone and tetrahydrofurfuryl methacrylate, hydroxyapatite, polyurethane, and polylactic acid. Besides freeze drying, other methods for drying the soft tissue scaffold or soft tissue composite include air drying or drying in a drying oven at a pre-set temperature.

The preparation method may further comprise adding one or more bioactive supplement(s) to the one or more soft tissue(s), the dispersed collagen fiber bundles, or the soft tissue scaffold and/or soft tissue composite. The preparation method may further comprise adding one or more bone, demineralized bone, or cartilage fragment material(s) to the one or more soft tissue(s), the dispersed soft tissue material, or the soft tissue scaffold and/or soft tissue composite.

### Example 1: Preparation of Collagen Fiber Bundle from Soft Tissue

Skin, fascia, and tendon from a human cadaver were procured and returned to the processing facility under sterile conditions. Donor histories, personal and medical, were obtained following accepted standards of the American Association of Tissue Banks. Microbiological tests were performed following FDA guidelines for testing sterility of products.

The skin pieces were cleaned of hair, unwanted adipose tissue and epithelial layer. The obtained dermis was treated with detergent containing N-Lauroylsarcosine and DNAase followed by saline rinse. The resulting cleaned and decellularized/devitalized dermis were used for the following experiments.

The fascia and tendon were cleaned of fat and muscle, followed by saline rinse. The fascia was lyophilized and used for the following experiments. The tendon was frozen until the following experiments.

### Method 1:

The soft tissue was cut into small (about 1.0-2.0 cm by 1.0-2.0 cm) pieces (e.g., crude fragments). About 24 grams of soft tissue and three ice cubes were mechanically dispersed (e.g., blended) together for 2 minutes using an Osterizer from Sunbeam-Oster, Inc., and three more pieces of ice cubes were added into the mixture and dispersed for another minute. Then the processed soft tissue was transferred onto a sieve. The undispersed tissue was picked out and mixed with two ice cubes and dispersed for another 2 minutes. Then the dispersed soft tissue was transferred back to the sterile sieve. The dispersed soft tissue containing collagen fiber bundle was collected and used for the following experiments.

### Method 2:

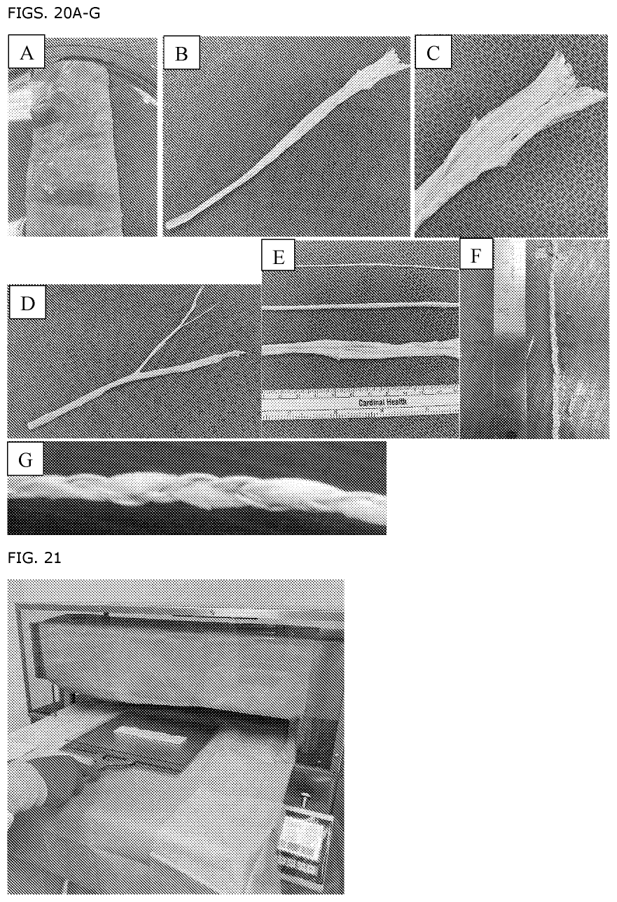

The cleaned fascia was rehydrated in isotonic saline and laid on a cutting board, the tissue was cut along the collagen fiber orientation as seen in FIG. 20A at the width of 2-3 mm. The resultant split collagen fiber bundle was either used directly in the following experiments or lyophilized for later use.

The cleaned tendon was thawed and laid on a surface, the fascicle bundles were split at the epitendon or peritendon. The resultant split collagen fiber bundles (fascicle bundles containing collagen fiber) was either used directly in the following experiments or lyophilized for later use (FIG. 20B-E).

### Example 2: Preparation of Mesh with Synthetic Polymers

### Method 1:

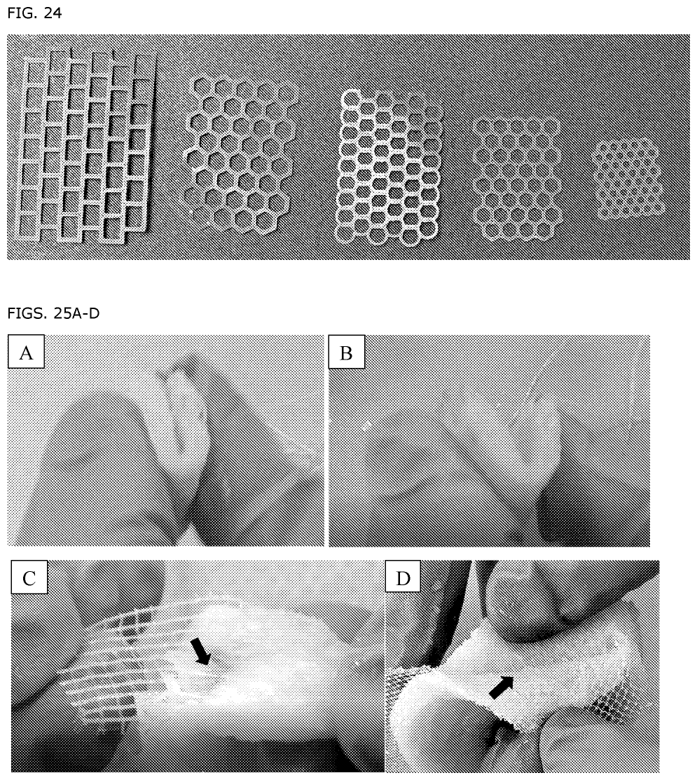

A 3D representation of the geometry was modeled in CAD software (i.e. SolidWorks) by creating a sketch of patterned geometric figures (i.e. rectangles, hexagons, etc.) and then extruding them to a 0.5 mm thickness. Various sizes and patterns were created. The solid 3D model was exported in STL (stereolithography) format and then imported into a slicing software (i.e. Dremel DigiLab 3D Slicer) for 3D printing. The layer height for printing was set at 0.1 mm per layer. The g-code (i.e. extruder toolpath) from the slicing software was exported and loaded into the fused filament 3D printer (Dremel 3D45) which takes a spool of 1.75 mm diameter filament (e.g. ECO-ABS, PETG, PLA, etc.) and extrudes it through a 0.4 mm diameter heated nozzle onto a heated print bed. After the print was complete, the parts were removed from the print bed (FIG. 24). Handling, strength, and bending properties were investigated by hand. A non-linear static finite element analysis was run on the printed geometry to compare trends in strength and stiffness versus geometry, and then also compared to the handing characteristics of the 3D printed parts.

### Method 2:

A representative 3D mesh was created by hand using a small diameter (0.09 mm) ultra-high molecular weight polyethylene braid and crochet techniques. Construction of the mesh began in the center and was constructed in a spiral manner outward. The circumferential expansion continued until the 3D construct matched the diameter of the silicon breast implant. Once this diameter was achieved additional layers were added to the outer diameter to give the mesh height. Large pores of approximately 1-2 mm in length and width were targeted. The mesh created is three dimensional in shape and cannot be laid flat (FIG. 8)

### Example 3: Preparation of Soft Tissue Scaffold

### Method 1:

The dispersed soft tissue containing collagen fiber bundles from example 1 was transferred into a mold and leveled to the surface of the mold. The molds containing dispersed collagen fiber bundles were mixed with a shaker or a mixer, stored at −20° C. or −80° C. freezer for a minimum of 4 hours, followed by freeze drying for 48-96 hours. After freeze-drying, the mold was removed and soft tissue scaffold was packaged and sent out for gamma-irradiation.

### Method 2:

The dispersed soft tissue containing collagen fiber bundles from example 1 was transferred into a mold and leveled to the surface of the mold. The molds containing dispersed soft tissue were mixed with a shaker or a mixer, stored at −20° C. or −80° C. freezer for a minimum of 4 hours, followed by freeze drying for 48-96 hours. After freeze-drying, the soft tissue scaffold was taken out of the mold and cut to the desired size, pressed using a SterilCut press (BioCut Systems, Inc.) with approximately 27 tons of force to a final thickness less than 1 mm (FIGS. 21 and 26 A-C). The soft tissue scaffold was packaged and sent out for gamma-irradiation.

### Example 4: Preparation of Soft Tissue Composite in a 2D Setting

Method 1: The dispersed soft tissue containing collagen fiber bundles from example 1 was transferred into a mold and leveled to the surface of the mold. A synthetic mesh (Vicryl, polypropylene) with pore size between 2-5 mm in diameter was cut to the size matching the mold size, laid on top of the first mold, and fixed at the 4 corners of the mold by screws. A second mold with the open bottom and top was added to the 1^{st} mold on the top of the mesh, aligned to the corner screws and secured by the screws. The dispersed soft tissue containing collagen fiber bundles from the example 1 was transferred into the 2^{nd} mold and leveled, the lid/closure was placed on the surface of the 2^{nd} mold. The entire assembly was secured by clamps and shaken on a shaker or a mixer for 3-5 minutes.

The molds containing dispersed soft tissue and synthetic mesh were stored at-20° C. or −80° C. freezer for a minimum of 4 hours, followed by freeze drying for 48-96 hours. After freeze drying, the mold was removed, and soft tissue was packaged and sent out for gamma-irradiation (FIG. 12A).

### Method 2:

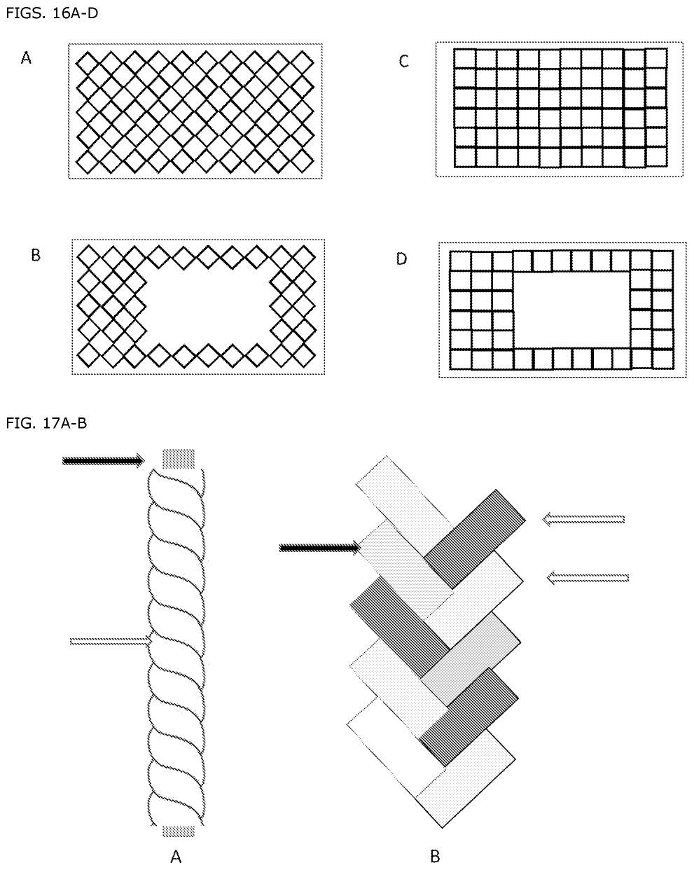

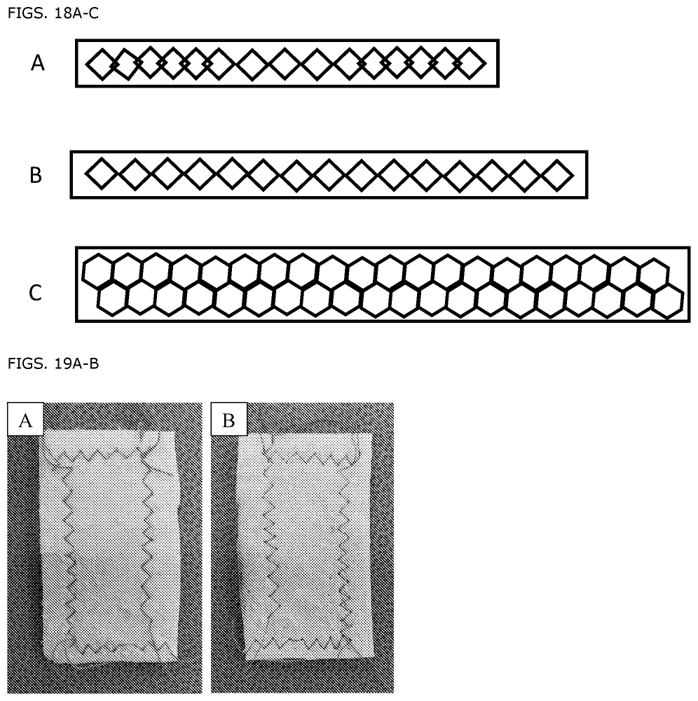

The soft tissue scaffold made from example 3 method 1 was taken out of the package and cut to the desired size. Strands made from resorbable or non-resorbable synthetic polymer were threaded into the needle. Stitches were made into the soft tissue scaffold either by hand sewing or sewing machine following different pattern (FIGS. 14A, 15, 16, and 19).

### Method 3:

The dispersed soft tissue from example 1 was transferred into a mold and leveled to the surface of the mold. The molds containing dispersed collagen fiber bundles were mixed with a shaker or a mixer, stored at −20° C. or −80° C. freezer for a minimum of 4 hours, followed by freeze drying for 48-96 hours. After freeze-drying, the mold was removed and soft tissue scaffold was packaged and sent out for gamma-irradiation.

The soft tissue scaffold was taken out of the package and cut to the desired size, pressed using a SterilCut press (BioCut Systems, Inc.) with approximately 27 tons of force to a final thickness less than 1 mm (FIGS. 21 and 26 A-C). Strands made from resorbable or non-resorbable synthetic polymer were threaded into the needle. Stitches were made into the soft tissue scaffold either by hand sewing or sewing machine following different pattern (FIG. 23A-B).

### Method 4:

A heating plate was used to heat the metal heating block to about 130° C. or 175° C. Place one side of the specimen made from method 1 on top of the heating block and place 200 g weight on top of the specimen and left the specimens on heat for about 10 seconds to 5 minutes. The specimens were taken out of the heating block and packaged for surface analysis.

### Example 5: Preparation of Soft Tissue Composite in a 3D Setting

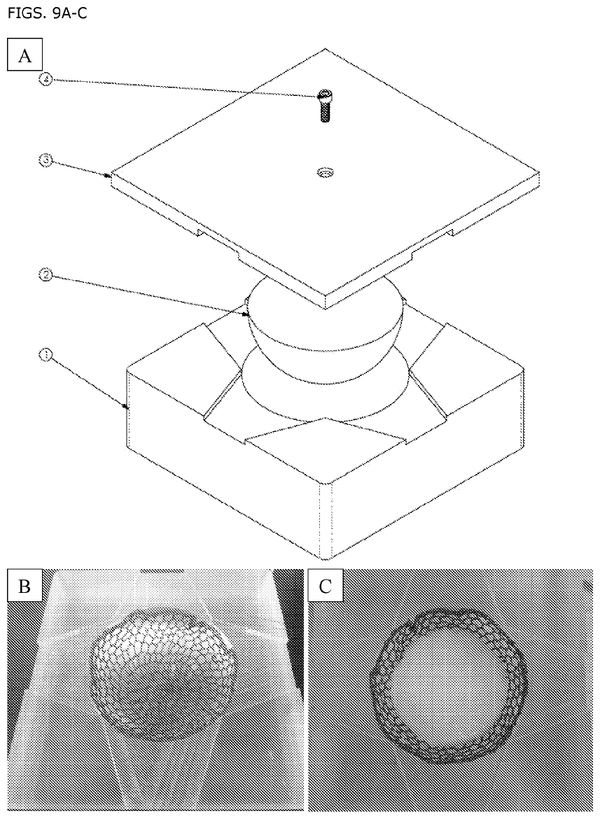

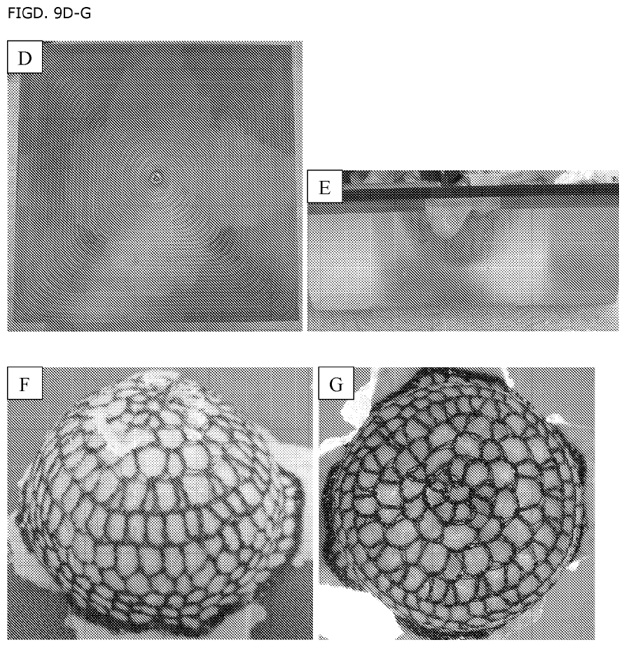

The 3D mesh made from Example 2 method 2 or a 2D meshed soft tissue such as skin or dermis was laid into a hemispherical mold base concentrically and conforming to the contour (FIG. 9B). The dispersed soft from example 1 was poured into the center of the hemispherical mold base partially covering the 3D mesh and filling the mold about half way (FIG. 9C). A second, smaller hemispherical mold insert piece attached to a lid was pressed into the dispersed soft tissue concentrically to the mold base until the mold lid rested on the top surface of the mold base (FIG. D-E). The soft tissue slurry filled the gap between the mold base and the mold insert. The mold assembly was frozen for a minimum of 4 hours, followed by freeze drying for 48-96 hours to give a rigid, 3D wrap with a laminated mesh. The thickness of the freeze dried soft tissue composite, not including the mesh, was 3-4 mm and the outer diameter was 12 cm (FIG. 9F-G)).

### Example 6: Preparation of Collagen Yarn with Soft Tissue

### Method 1:

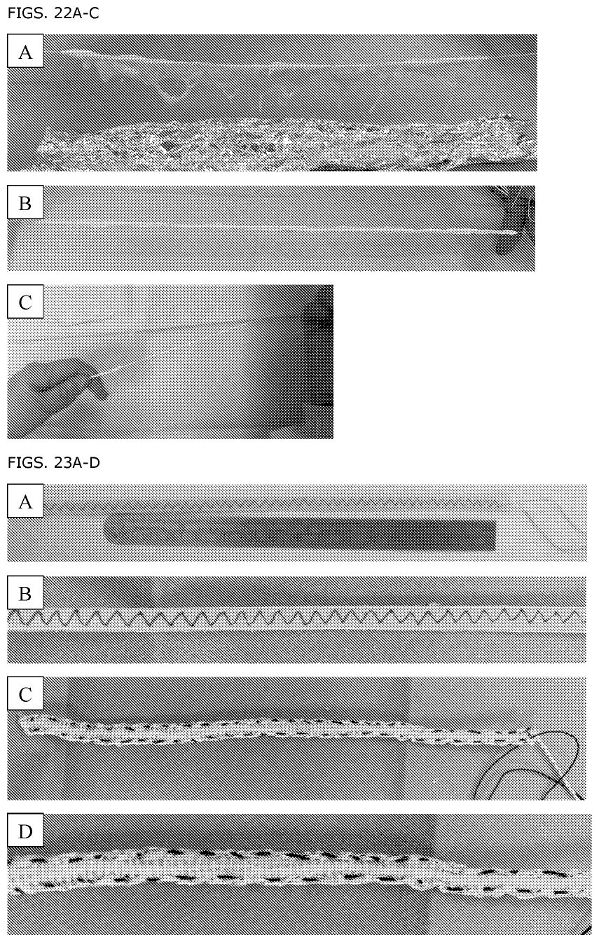

The dispersed soft tissue containing collagen fiber bundles from example 1 was transferred into a boat made from foldable material such as aluminum foil with wide opening. A synthetic strand made from either resorbable or non-resorbable polymers was laid on top of the dispersed collagen fiber bundles and holding straight and tight at both ends, and the boat was closed by folding and the entire boat containing dispersed collagen fiber bundles and synthetic strand was moved and turned around the fixed strand at both ends. After opening the boat, the suture coated with dispersed soft tissue was trimmed of excess collagen fibre bundles (FIGS. 22A-B), optionally passed through an open tubing which is about 4-5 mm in diameter. The strand coated with collagen fiber bundles was either suspended on a box or laid in the mold. The composite was stored at −20° C. or −80° C. freezer for a minimum of 4 hours, followed by freeze drying for 48-96 hours.

After removing out of the drier, the collagen fiber bundles were stripped off from the strand from one end as one piece (FIG. 22C). This piece of dispersed soft tissue was pressed to a compact strand of collagen yarn.

### Example 7: Preparation of Composite Strand or Ribbon with Collagen Fiber Bundles and Polymeric Material

### Method 1:

The dispersed soft tissue containing collagen fiber bundles from example 1 was transferred into a boat made from foldable material such as aluminum foil with wide opening. A synthetic strand or suture without needle made from either resorbable or non-resorbable polymers was laid on top of the dispersed soft tissue and holding straight and tight at both ends, and the boat was closed by folding and the entire boat containing dispersed soft tissue and synthetic strand or suture was moved and turned around the fixed strand or suture at both ends. After opening the boat, the strands or suture coated with dispersed soft tissue was trimmed of excess collagen fiber bundles (FIGS. 22A-B). The strand or suture coated with collagen fiber bundles was either suspended on a box or laid in the mold. The composite strand or suture was stored at-20° C. or −80° C. freezer for a minimum of 4 hours, followed by freeze drying for 48-96 hours.

After freeze drying, the composite strand or suture was wrapped with a membrane tightly, either rolled it with a rolling pin or pressed using a press (BioCut Systems, Inc.) with approximately 27 tons of force to a final diameter less than 1 mm.

### Method 2:

The soft tissue scaffold made from method 2 of example 3 was cut to slices in the width between 2-3 mm. A suture tape made from non-resorbable polymers was folded in half of length. Strands made from non-resorbable synthetic polymer were threaded into the needle. Stitches was placed at one edge of the folded suture tape to form a pocket. Soft tissue scaffold was cut to the size that fit the folded suture tape and placed into the pocket, then stitches was placed on the other side of the pocket to encase the soft tissue scaffold (FIGS. 23C-D).

### Method 3:

The soft tissue scaffold made from example 3 method 2 was taken out of the package and cut to the desired size. Strands made from resorbable or non-resorbable polymer were threaded into the needle. Stitches were made into the soft tissue scaffold either by hand sewing or sewing machine following different pattern (FIGS. 23A-B).

### Method 4:

The composite strand or suture made from method 1 is braided or weaved with more strands that are made of composite strand, suture, or ribbon.

### Method 5:

The split collagen fiber bundles made from Example 1 method 2 was braided or weaved with more strands that were made of resorbable or non-resorbable polymeric material (FIGS. 20 F and G).

### Example 8: Characterization of the Soft Tissue Composite

Suture retention was performed on the specimen made from Example 4 method 1 using Instron (Model 3367) and load cell (2530-500N). Each sample was cut to 2 cm x 3-4 cm rectangular shape. Suture (FiberWire 0) was placed at about 5 mm from the edge of short side of the rectangular shape to test long axis suture retention. Two double overhands then 2 single overhands loops were tied. The suture was hung over cross bar on the upper fixture and clamped at least 1 cm of the specimen in the clamp on the bottom fixture. The specimen was pulled at 20 mm/min and the test ended after 90% drop from the maximum force. The maximum load at break was recorded. The average of the maximum load at break from the specimen made with resorbable polymer mesh was 26.02N and the maximum load at break from the specimen made with non-resorbable polymer mesh was 22.08N.

Tensile property was measured on the specimen made from the Example 4 method 1 with the Instron (Model 3367) and load cell (5 kN 2530-405). The specimens were cut to about 2 cm×5-6 cm size and the thickness of the specimens was measured. The setting of ramp was 20 mm/min, end test activation level was 20N, and the end test value measured when 90% drop from the maximum force. The maximum load and stiffness from the specimen made with resorbable polymer mesh was 133.43N at long axis and 8802.8 N/m, respectively. The maximum load and stiffness from the specimen made with non-resorbable polymer mesh was 48.3N and 21.5 N/m, respectively.

### Integration Test:

Samples made from Example 4 was tested for the integration of both layers of soft tissue scaffolds and polymer mesh with 2 different methods. The first method used load cell (Futek 250 lb compression button type) and Sensit software. The specimen was cut to 2 cm×3 cm size, sutured (FiberWire 0) at 4 corners, pre-load to an inserter device (Arthrex Inc.), and hydrated the specimen in saline before testing. The pre-loaded device was placed on top of the load cell (FIG. 27A) and passed through a surgical cannular (Arthrex Passport 10 mm×3 cm) (FIG. 27B). The force used to pass through the cannular was recorded. The specimen was rehydrated with saline and examined for any separation between soft tissue scaffolds and polymeric mesh. The average force of 4 tests was 18.66±1.8N and the highest force was 20.79N. After rehydration no separation of the soft tissue scaffolds and polymeric mesh was observed.

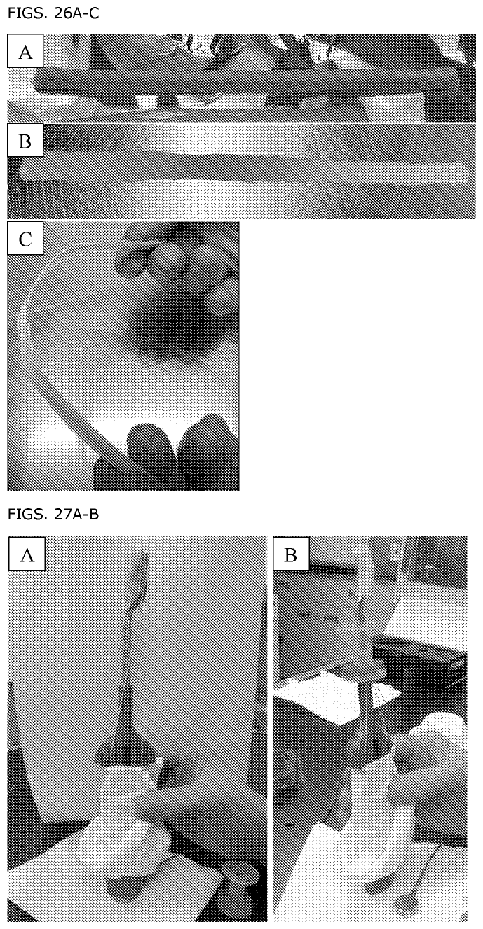

The second method used to evaluate the integration of the soft tissue scaffolds with the polymeric mesh was separating the layers by fingers or forceps. The specimen was cut to 2 cm×3 cm size and hydrated with saline. The specimens were folded by hands to check the separation of layers (FIGS. 25A-B). Then one or both soft tissue scaffold layers of the composite specimen was peeled off from polymeric mesh with fingers to examine the connection between the soft tissue scaffold and polymer mesh (FIGS. 25C-D). Collagen fiber bundles were found connecting one layer of soft tissue scaffold with the polymeric mesh or the collagen fiber bundles from other layer of soft tissue scaffold.

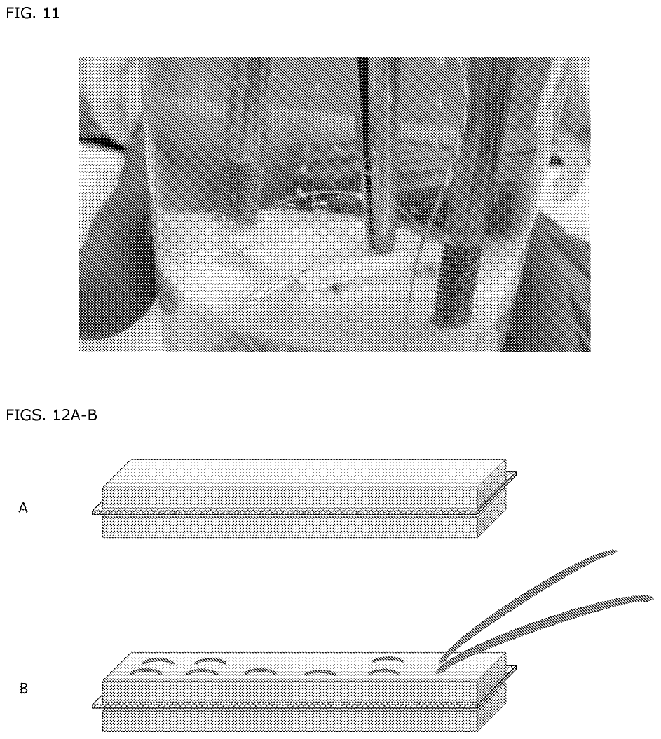

The handling of the specimen made from Example 4 method 4 was examined with relevant surgical instruments (FIG. 10). The specimen was sutured at four corners, fixed to an insert device (Arthrex Inc.) with the surface treated side facing up, hydrated with saline, and passed through a cannular (Arthrex Passport 10 mm×3 cm) (FIG. 10). The surface characteristics of the specimen made from Example 4 method 4 was examined with an in vivo simulating device (FIG. 11). After the specimen removed from the insertion device, it was fixed on a stage with the surface treated side facing up, transferred the stage into a chamber, filled with saline. The chamber was also connected to a peristatic pump to provide the saline flow (150 ml/min) into the chamber constantly for a minimum of 30 minutes. Rough manipulation of the specimen with forceps was performed during this time. Any collagen fiber bundle dissociation from the specimen was observed and recorded (FIG. 11).

### Example 9: Preparation of Tissue Repair Implants with 2D Soft Tissue Composite

To prepare for the arthroscopic implantation, the graft from the Example 4 method 4 were pre-sutured at four corners with appropriate suture types, pre-loaded to an insertion device with the surface treated side facing up and packaged. At the time of surgery using cadaveric shoulder, the surgeon opened the package, rehydrated the soft tissue composite with saline for less than 5 minutes, and passed the soft tissue composite through the cannular and laid flat on the rotator cuff closed to the lateral side, fixed the lateral sutures with bone suture anchors and the medial side with the Fibertak soft tissue knotless anchors. The surface of the soft tissue composite looked smooth without dissociated collagen fiber bundles under arthroscopic camera.

### Example 10: Preparation of Tissue Repair Implants with 3D Soft Tissue Composite

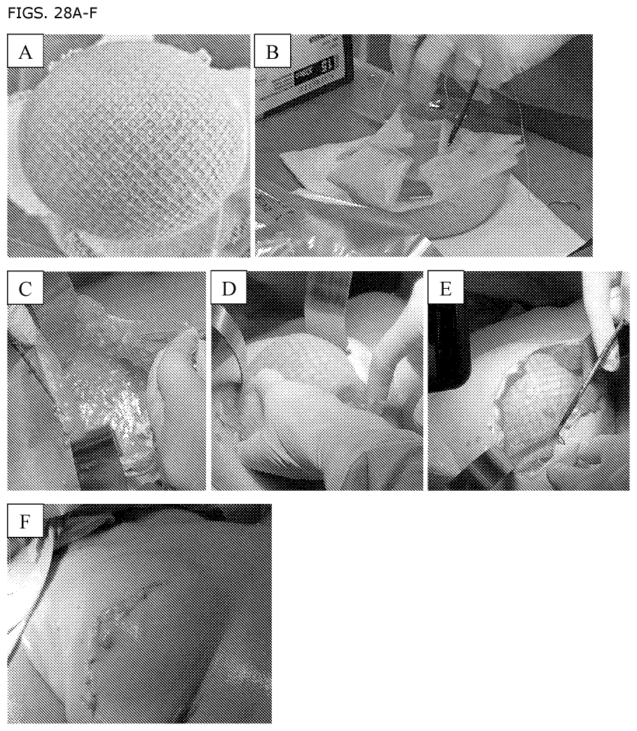

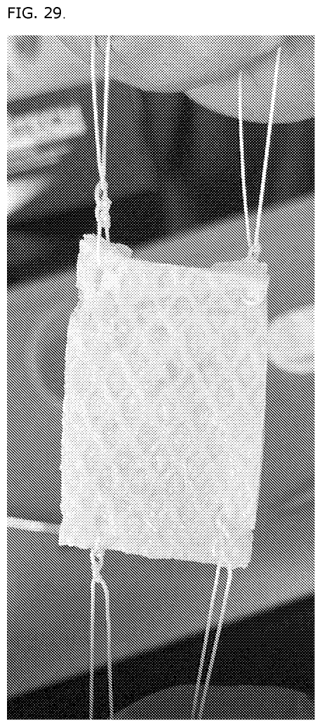

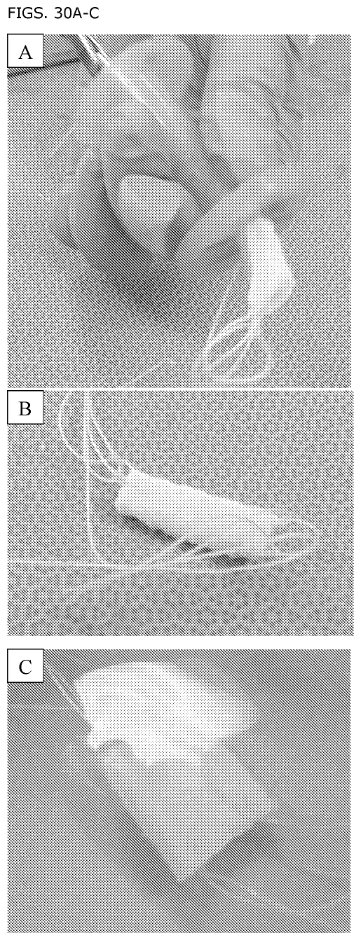

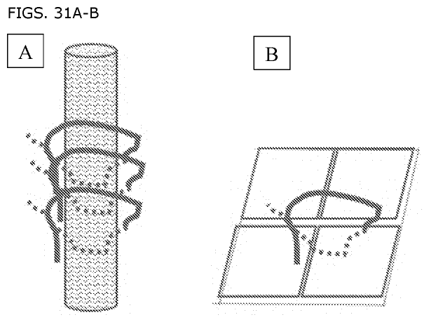

The soft tissue composite made from Example 5 was taken out from the package (FIG. 28A), hydrated with saline for 5 minutes, and placed upside down on the table. The breast implant was placed in the pocket of the 3D soft tissue composite and the sutures were made to wrap the implant with the soft tissue composite (FIG. 28B). A mastectomy was performed in a Cadaver right breast (FIG. 28C), 3D soft tissue composite wrapped breast implant was placed inside the skin pocket (FIG. 28D) and sutured to the chest muscle at the inferior side and lateral side (FIG. 28E). Followed by the skin closure, the breast implant was positioned and stayed at the space (FIG. 28F).

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A soft tissue composite, comprising a first collagen fiber bundle of a first soft tissue isolated from a first donor and a polymeric material, wherein the first collagen fiber bundle interconnects with the polymeric material.

2. The soft tissue composite of claim 1, wherein the polymeric material is entangled with the first collagen fiber bundle.

3. The soft tissue composite of claim 1, wherein the polymeric material comprises pores, and the first collagen fiber bundle passes through the pores.

4-10. (canceled)

11. The soft tissue composite of claim 1, wherein the first collagen fiber bundle is selected from the group consisting of a first split collagen fiber bundle, a first dispersed collagen fiber bundle and a combination thereof.

12. The soft tissue composite of claim 11, wherein the first dispersed collagen fiber bundle is in the form of a first soft tissue scaffold or a first collagen yarn.

13. (canceled)

14. The soft tissue composite of claim 1, wherein the polymeric material is in the form of a polymer strand, a polymer ribbon or a polymer mesh.

15-25. (canceled)

26. The soft tissue composite of claim 1, further comprising an additional collagen fiber bundle.

27. (canceled)

28. The soft tissue composite of claim 26, wherein the composite strand or ribbon forms a composite mesh, and wherein the additional dispersed collagen fiber bundle and the composite mesh form a composite scaffold.

29. The soft tissue composite of claim 1, wherein the soft tissue composite comprises a first layer, wherein the first layer comprises the first collagen fiber bundle.

30-35. (canceled)

36. The soft tissue composite of claim 29, wherein the soft tissue composite further comprises a second layer, wherein the second layer comprises the polymeric material in the form of a polymer mesh.

37. The soft tissue composite of claim 36, wherein the first layer covers the second layer.

38-50. (canceled)

51. The soft tissue composite of claim 36, wherein the soft tissue composite further comprises a third layer, wherein the second layer is between the first layer and the third layer, and the third layer comprises a third collagen fiber bundle of a third soft tissue isolated from a third donor.

52. The soft tissue composite of claim 51, wherein the third collagen fiber bundle is in the form of a third soft tissue scaffold.

53. The soft tissue composite of claim 52, wherein the polymer mesh has pores, and the first soft tissue scaffold and the third soft tissue scaffold interconnect with each other through the pores.

54. The soft tissue composite of claim 52, wherein the polymeric material is entangled with the third collagen fiber bundle.

55-58. (canceled)

59. The soft tissue composite of claim 51, wherein the second layer further comprises a second collagen fiber bundle of a second soft tissue isolated from a second donor.

60-73. (canceled)

74. The soft tissue composite of claim 1, wherein pressed and hydrated soft tissue composite is capable of regaining the original shape and thickness after the pressing force is removed.

75. A medical device comprising the soft tissue composite of claim 1.

76. (canceled)

77. A method of repairing a defective tissue in a subject in need thereof, comprising administering the soft tissue composite of claim 1 to the defective tissue in the subject.

78-81. (canceled)

82. A method of preparing the soft tissue composite of claim 1, comprising interconnecting the first collagen fiber bundle with the polymeric material.

83-98. (canceled)

99. A method of preparing a soft tissue composite, comprising:
(a) dispersing a soft tissue to make dispersed collagen fiber bundles;
(b) covering a top surface of a first mold with the dispersed collagen fiber bundles to form a first layer, wherein the first mold has a rim;
(c) placing a polymer mesh on the top of the first layer to form the second layer, wherein the polymer mesh has an edge larger than the rim of the first mold; and
(d) fixing the edge of the polymer mesh to the rim of the first mold, whereby a soft tissue composite is obtained.

100. A method of preparing a soft tissue composite, comprising:
(a) dispersing a soft tissue to make dispersed collagen fiber bundles;
(b) covering the surface of a first mold with a polymer mesh;
(c) transfer the dispersed collagen fiber bundles on top of the mesh;
(d) inserting a second mold with a lid into the dispersed collagen fiber bundles; and
(e) removing extra dispersed collagen fiber bundles from the rim of the second mold, whereby a soft tissue composite is obtained.

101-108. (canceled)
